(19) United States
(12) Patent Application Publication
(10) Pub. No.: US 2014/0081142 A1
(43) Pub. Date: Mar. 20, 2014

(54) ULTRASOUND DIAGNOSTIC APPARATUS AND CONTROL METHOD FOR ULTRASOUND DIAGNOSTIC DEVICE

(71) Applicant: Panasonic Corporation, Osaka (JP)

(72) Inventors: Tadamasa TOMA, Osaka (JP); Jun OHMIYA, Kyoto (JP); Bumpei TOJI, Gifu (JP)

(73) Assignee: Panasonic Corporation, Osaka (JP)

(21) Appl. No.: 14/085,055

(22) Filed: Nov. 20, 2013

Related U.S. Application Data
(63) Continuation of application No. PCT/JP2013/002728, filed on Apr. 23, 2013.

(30) Foreign Application Priority Data
Apr. 23, 2012 (JP) 2012-097387

(51) Int. Cl. A61B 8/00; A61B 8/08

(52) U.S. Cl. CPC A61B 8/4245; A61B 8/0891; A61B 8/5207
USPC 600/443

(57) ABSTRACT

An ultrasound diagnostic apparatus includes a measurement position-orientation determination unit, a judgment unit and a property measurement unit. The measurement position-orientation determination unit determines a measurement target region for measuring a property of a vascular wall, based on a 3D image of a blood vessel generated from transverse tomographic images acquired by scanning of an ultrasound probe in a longitudinal direction, and also determines a measurement position and measurement orientation of the ultrasound probe for acquiring a longitudinal tomographic image including the measurement target region. The judgment unit judges whether a current position and current orientation of the ultrasound probe differ from the measurement position and measurement orientation respectively by no greater than a threshold value, and when judging affirmatively causes the apparatus to acquire the longitudinal tomographic image. The property measurement unit measures the property based on the longitudinal tomographic image.

### CROSS REFERENCE TO RELATED APPLICATION

This is a continuation application of PCT Application No. PCT/JP2013/002728 filed Apr. 23, 2013, designating the United States of America, the disclosure of which, including the specification, drawings and claims, is incorporated herein by reference in its entirety.

### TECHNICAL FIELD

The present disclosure relates to an ultrasound diagnostic apparatus and control method thereof that analyze ultrasound images to automatically determine a position of a measurement target, and guide operation by an operator in a manner such that an ultrasound image of the position can be acquired.

### DESCRIPTION OF THE RELATED ART

Diagnostic imaging apparatuses which are commonly used on living organisms include X-ray diagnostic apparatuses, MRI (Magnetic Resonance Imaging) diagnostic apparatuses and ultrasound diagnostic apparatuses. Among the above apparatuses, ultrasound diagnostic apparatuses in particular have advantages in terms of non-invasiveness and real time usage, and therefore are widely used in health examinations and diagnosis. An ultrasound diagnostic apparatus may be used on various diagnostic targets such as blood vessels, heart, liver and breasts. In particular, in recent years there has been interest in using examination of the carotid artery in order to determine risk of arteriosclerosis.

The following explains examination of the carotid artery using an ultrasound diagnostic apparatus. FIGS. 19A-19D are provided for explaining an image viewed when scanning the carotid artery with the ultrasound diagnostic apparatus. FIG. 19A illustrates an ultrasound probe. A plurality of ultrasound transducers are arranged in a column on the ultrasound probe. Herein, the column of ultrasound transducers is referred to as a transducer column When ultrasound transducers are arranged in a 1D (one dimensional) array, such as in the present example, an ultrasound image is obtained of a 2D (two dimensional) scan plane directly below the ultrasound transducers. As illustrated in FIG. 19B, in examination of the carotid artery, images are acquired which are viewed in a direction in which the carotid artery extends (herein referred to as a longitudinal direction) and also images are acquired which are viewed in a direction which is roughly perpendicular to both the longitudinal direction and a depth direction of the skin (herein referred to as a transverse direction). When the ultrasound probe is scanned along the carotid artery in the transverse direction, longitudinal tomographic images are acquired which are cross-section images along a longitudinal axis of the carotid artery, such as illustrated in FIG. 19C. On the other hand, when the ultrasound probe is scanned along the carotid artery in the longitudinal direction, transverse tomographic images are acquired which are cross-section images that cut across the carotid artery in the transverse direction, such as illustrated in FIG. 19D.

The following explains structure of the carotid artery. FIG. 20 is a perspective diagram illustrating structure of the carotid artery along the longitudinal direction thereof. As illustrated in FIG. 20, the carotid artery includes a common carotid artery (CCA) 213 which configures a central end of the carotid artery, and an internal carotid artery (ICA) 215 and an external carotid artery (ECA) 216 which configure a peripheral end of the carotid artery. A bulb of the common carotid artery (bulb) 214 is located between the CCA 213, and the ICA 215 and the ECA 216. Also, a bifurcation of the common carotid artery (Bif) 217 is located at a branching point from the bulb 214 to the ICA 215 and the ECA 216.

The following explains structure of a vascular wall. FIGS. 21A-21C are schematic diagrams illustrating structure of a vascular wall of an artery. As illustrated in FIGS. 21A and 21B, the vascular wall of the artery is configured by three layers. In an outward direction from a lumen of the artery, the three layers are referred to as a tunica intima, a tunica media and a tunica adventitia. An interface between the lumen and the tunica intima is referred to as a lumen-intima interface and an interface between the tunica media and the tunica adventitia is referred to as a media-adventitia interface.

In carotid artery examination, thickness of the vascular wall is used as an indicator of a degree of progression of arteriosclerosis. As arteriosclerosis progresses, hypertrophy occurs mainly of the tunica intima and the tunica media. Consequently, in examination of the carotid artery using ultrasound, thickness of an intima-media complex consisting of the tunica intima and the tunica media, referred to as an intima-media thickness (IMT), is measured by detecting the lumen-intima interface and the media-adventitia interface. A state in which hypertrophy of the intima-media complex causes the IMT to exceed a predetermined value in a localized area is referred to as a plaque. FIG. 21C illustrates change in structure of the vascular wall in the above state. Depending on size of the plaque, treatment may be necessary such as medicinal treatment or surgical treatment to remove the plaque. Consequently, accurate measurement of intima-media thickness is vital in diagnosis of arteriosclerosis.

However, intima-media thickness varies depending on a part of the carotid artery which is measured. Also, it is difficult for an examiner to grasp 3D (three dimensional) shape of the carotid artery due to the carotid artery being concealed in the neck, thus examination of the carotid artery requires a high degree of procedural skill. In regards to the above, methods have been proposed for carotid artery examination in which measurement is automated, thus removing the need for a high degree of procedural skill. For example, Patent Literature 1 proposes an art in which a plurality of tomographic images acquired by manually scanning an ultrasound probe along a longitudinal direction of a carotid artery are used to construct a 3D image of the carotid artery, and subsequently a longitudinal cross-section image is extracted from the 3D image for use in IMT measurement. The method disclosed in Patent Literature 1 is explained with reference to FIG. 22.

FIG. 22 is a schematic diagram illustrating a method of constructing a 3D image of the carotid artery. A scan is first performed in the longitudinal direction along the entire length of the carotid artery, thus acquiring a plurality of transverse tomographic images (FIG. 22, section (a)). Vascular contours of the carotid artery are extracted from each of the transverse tomographic images (FIG. 22, section (b)). Next, the vascular contours in each frame are arranged in a 3D space (FIG. 22, section (c)), and a 3D image of the carotid artery is constructed by for example generating polygons based on apices of the vascular contours (FIG. 22, section (d)). Finally, the 3D image is analyzed to extract a longitudinal cross-section image along a vascular central axis for use in IMT measurement.

### CITATION LIST

### Patent Literature

### [Patent Literature 1]

Japanese Patent Application Publication No. 2003-305039

### SUMMARY

### Technical Problem

Due to the nature of arteriosclerosis, regular performance of IMT measurement is necessary, and for purposes of accurate diagnosis, preferably IMT measurement should be performed at the same position during each measurement. Patent Literature 1 discloses a method for extracting, from a 3D image of the carotid artery, a longitudinal cross-section image for IMT measurement, along a vascular central axis of a carotid artery. However, Patent Literature 1 does not disclose an art which can accurately determine a position and an orientation of an ultrasound probe which are appropriate for IMT measurement.

Consequently, in the conventional art an operator is required to manually perform operations to determine a measurement target region for IMT and to guide the ultrasound probe to the measurement target region. Consequently, measurement is difficult if the operator does not possess a high degree of procedural skill and increased examination time is required in order to ensure accurate measurement.

In light of the above problem, the present disclosure aims to provide an ultrasound diagnostic apparatus and a control method thereof which allow an operator to perform IMT measurement easily, even if the operator does not possess a high degree of procedural skill.

### Solution to Problem

In order to solve the above problem, in one general aspect of the present disclosure an ultrasound diagnostic apparatus, to which an ultrasound probe and a position-orientation detection unit, configured to detect a position and an orientation of the ultrasound probe, are connectable, comprises: a transmission-reception processing unit configured to transmit an ultrasound wave toward a blood vessel which is a measurement target via the ultrasound probe and to receive a reflected ultrasound wave from the blood vessel via the ultrasound probe; a 2D image generation unit configured to generate a tomographic image of the blood vessel based on the reflected ultrasound wave; a measurement position-orientation determination unit configured to determine a measurement target region of the blood vessel, based on a 3D image of the blood vessel generated from a plurality of transverse tomographic images acquired by scanning of the ultrasound probe along the blood vessel in a longitudinal direction, and to determine a measurement position and a measurement orientation of the ultrasound probe for acquiring a longitudinal tomographic image including the measurement target region; a judgment unit configured to judge whether a current position and a current orientation of the ultrasound probe, detected by the position-orientation detection unit at a current time, differ from the measurement position and the measurement orientation respectively by no greater than a threshold value; and a property measurement unit configured to calculate a property of a vascular wall of the blood vessel in the measurement target region, wherein when the judgment unit judges that the current position and the current orientation differ from the measurement position and the measurement orientation respectively by no greater than the threshold value, the property measurement unit calculates the property of the vascular wall based on the longitudinal tomographic image of the blood vessel.

### Advantageous Effects of Invention

The ultrasound diagnostic apparatus relating to the present disclosure can be easily operated, even by an operator who does not possess a high degree of procedural skill, to quickly perform IMT measurement, and thus accuracy and reproducibility of IMT measurement by the operator who does not possess a high degree of procedural skill can be improved.

### BRIEF DESCRIPTION OF DRAWINGS

FIG. 1 is a block diagram illustrating configuration of an ultrasound diagnostic apparatus 10 relating to a first embodiment.

FIGS. 2A-2E illustrate an overview of functions of the ultrasound diagnostic apparatus 10 relating to the first embodiment.

FIG. 3 is a flowchart illustrating operations of the ultrasound diagnostic apparatus 10 relating to the first embodiment.

FIGS. 4A-4C are provided for explaining operations of the ultrasound diagnostic apparatus 10, relating to the first embodiment, in Step S202.

FIG. 5 illustrates a display example of navigation information in the ultrasound diagnostic apparatus 10 relating to the first embodiment.

FIG. 6 is a block diagram illustrating functional configuration of an ultrasound diagnostic apparatus 20 relating to a second embodiment.

FIGS. 7A and 7B are overview diagrams provided for explaining a method for combining results of analysis of transverse and longitudinal tomographic images in the ultrasound diagnostic apparatus 20 relating to the second embodiment.

FIG. 8 is a flowchart illustrating operations of the ultrasound diagnostic apparatus 20 relating to the second embodiment.

FIGS. 9A-9C are diagrams provided for explaining a relationship between a longitudinal tomographic image and a measurement target region in the ultrasound diagnostic apparatus 20 relating to the second embodiment.

FIG. 10A is a flowchart illustrating determination of a measurement position and a measurement orientation during IMT measurement by the ultrasound diagnostic apparatus 20 relating to the second embodiment, and FIG. 10B is a flowchart illustrating determination of a measurement position and a measurement orientation during Max-IMT measurement by the ultrasound diagnostic apparatus 20 relating to the second embodiment.

FIG. 11 is a block diagram illustrating configuration of an ultrasound diagnostic apparatus 30 relating to a third embodiment.

FIGS. 12A-12C are diagrams provided for explaining a method for measuring plaque volume in the ultrasound diagnostic apparatus 30 relating to the third embodiment.

FIG. 13 is a flowchart illustrating operations of the ultrasound diagnostic apparatus 30 relating to the third embodiment.

FIG. 14 is a block diagram illustrating configuration of an ultrasound diagnostic apparatus 40 relating to a fourth embodiment.

FIG. 15 is a schematic diagram illustrating an ultrasound probe 91 used by the ultrasound diagnostic apparatus 40 relating the fourth embodiment.

FIG. 16 is a flowchart illustrating operations of the ultrasound diagnostic apparatus 40 relating to the fourth embodiment.

FIG. 17 is a schematic diagram illustrating an ultrasound probe 92 used by an ultrasound diagnostic apparatus 40A relating to a modified example of the fourth embodiment.

FIGS. 18A-18C are provided for explaining a configuration in which a control method for an ultrasound diagnostic apparatus relating to a fifth embodiment is implemented by a computer system executing a program recorded on a recording medium such as a floppy disk.

FIGS. 19A-19D are provided for explaining an image which is viewed when using an ultrasound diagnostic apparatus to scan a carotid artery.

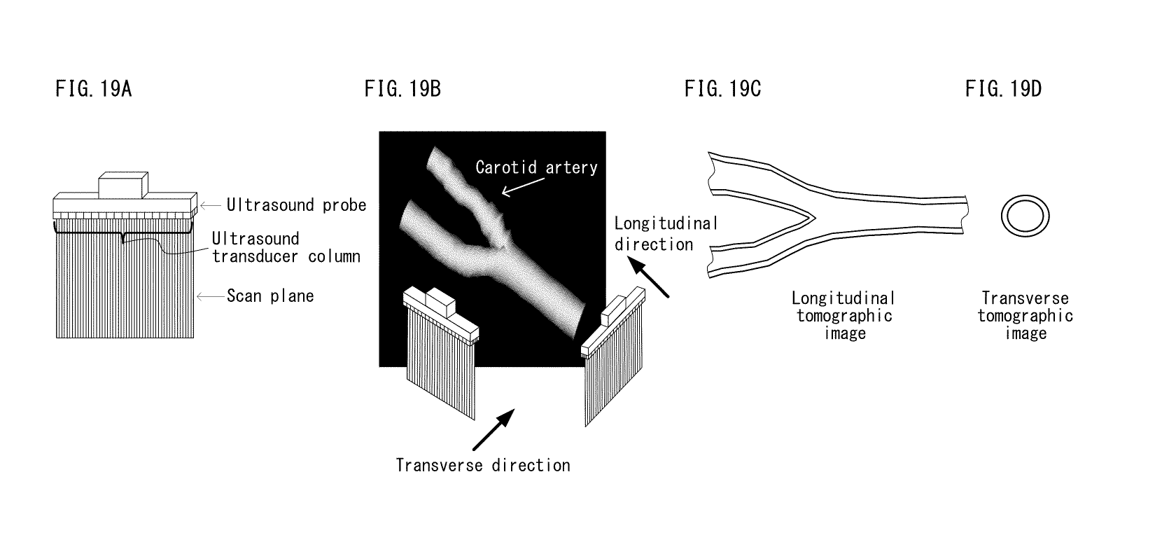

FIG. 20 is a perspective diagram illustrating structure of the carotid artery along a longitudinal direction thereof.

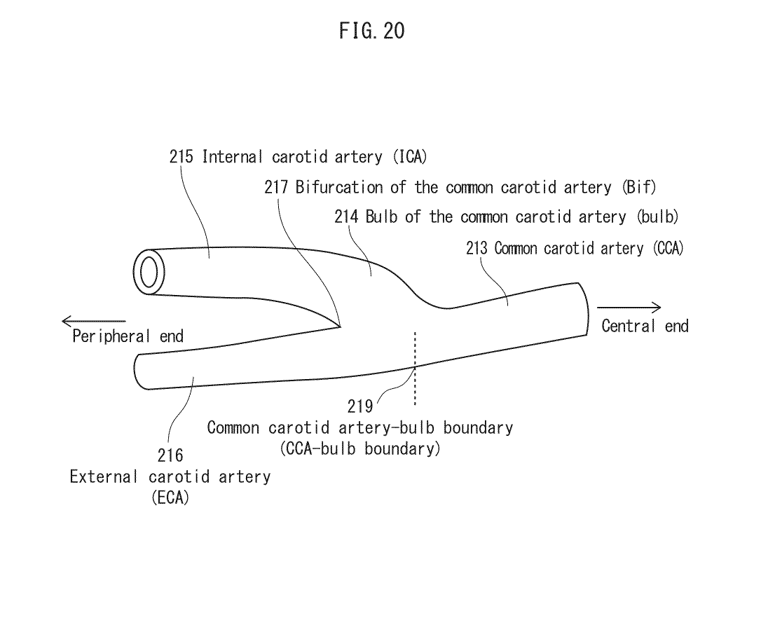

FIGS. 21A-21C are schematic diagrams illustrating structure of a vascular wall of an artery.

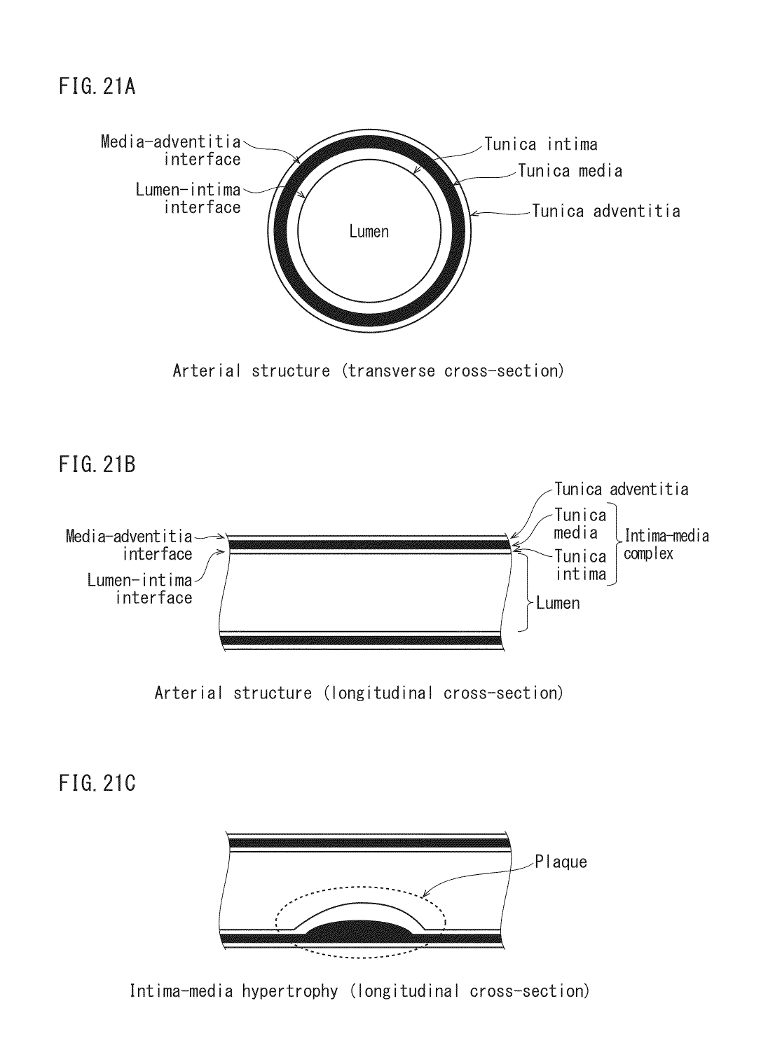

FIG. 22 is a schematic diagram illustrating a method of constructing a 3D image of the carotid artery in a conventional ultrasound diagnostic apparatus.

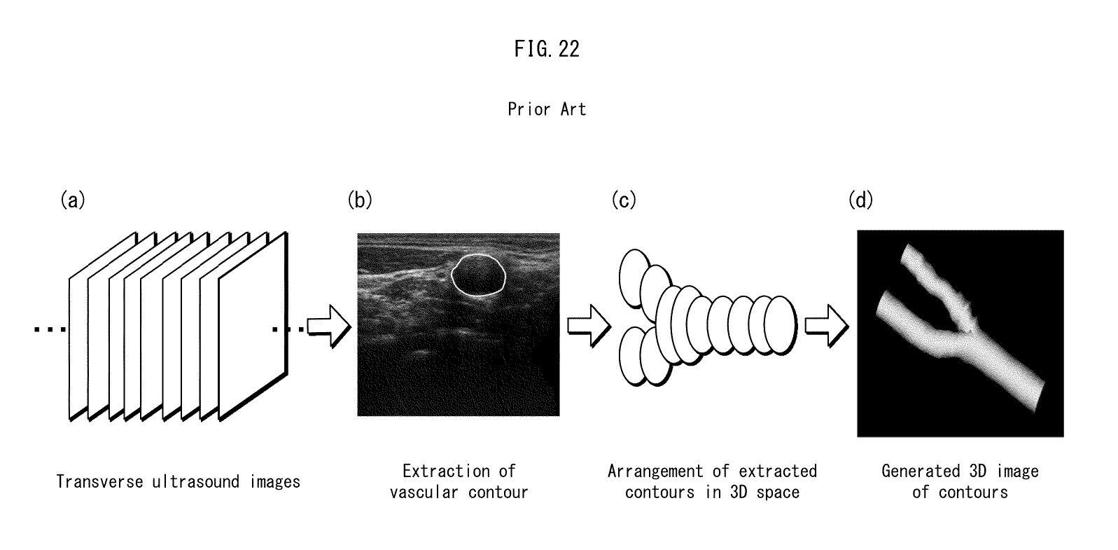

FIGS. 23A-23C are schematic diagrams for explaining a method envisaged by the inventors for defining a position and an orientation of a cross-section of a measurement target blood vessel, at which an ultrasound image appropriate for IMT measurement can be acquired by an ultrasound diagnostic apparatus.

FIG. 24 is a block diagram illustrating configuration of an ultrasound diagnostic apparatus 00 envisaged by the inventors.

FIG. 25 is a flowchart illustrating operations of the ultrasound diagnostic apparatus 00 envisaged by the inventors.

FIGS. 26A and 26B illustrate a longitudinal cross-section image extracted from a transverse 3D image which is generated from a plurality of transverse tomographic images acquired by scanning along the carotid artery in the longitudinal direction using the ultrasound diagnostic apparatus 00 envisaged by the inventors.

### DETAILED DESCRIPTION

### Background Leading to Embodiments of Present Disclosure

The inventors performed wide ranging research into an ultrasound diagnostic apparatus for determining a measurement target region for IMT.

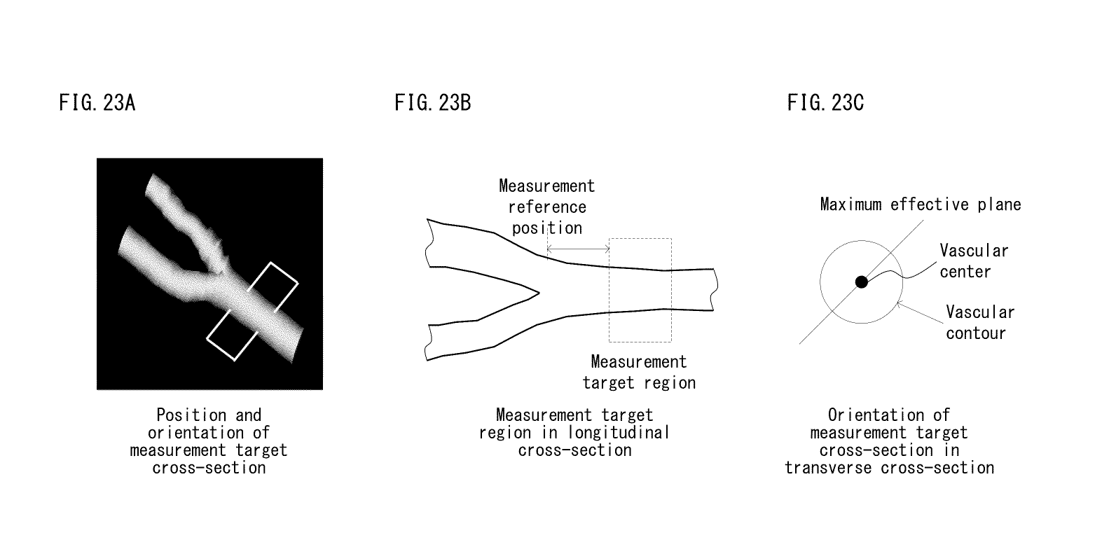

FIGS. 23A-23C are schematic diagrams for explaining, in an ultrasound diagnostic apparatus envisaged by the inventors, a method for defining a position and an orientation of a cross-section of a target blood vessel, at which an ultrasound image appropriate for IMT measurement can be acquired. FIG. 23A illustrates on a 3D image, the position and the orientation of the cross-section of the blood vessel, at which the ultrasound image appropriate for IMT measurement can be acquired. A measurement position and a measurement orientation may vary depending on an objective of examination. For example, as illustrated in FIG. 23B, during IMT measurement for diagnosis of arteriosclerosis, a measurement target region is defined in terms of longitudinal direction of the carotid artery as a region which is a predetermined distance from a measurement reference point, set based on external shape of the carotid artery. In terms of transverse direction, as illustrated in FIG. 23C, a cross-section which is a measurement target is defined as an arbitrary flat plane (hereinafter referred to as a maximum effective plane) containing a line (hereinafter referred to as a central line) which joins center points of vascular contours extracted from each of the transverse tomographic images configuring the 3D image. An orientation of the maximum effective plane of the transverse tomographic images is input by an operator. IMT is calculated by analyzing the 3D image in the measurement target cross-section which is determined as described above.

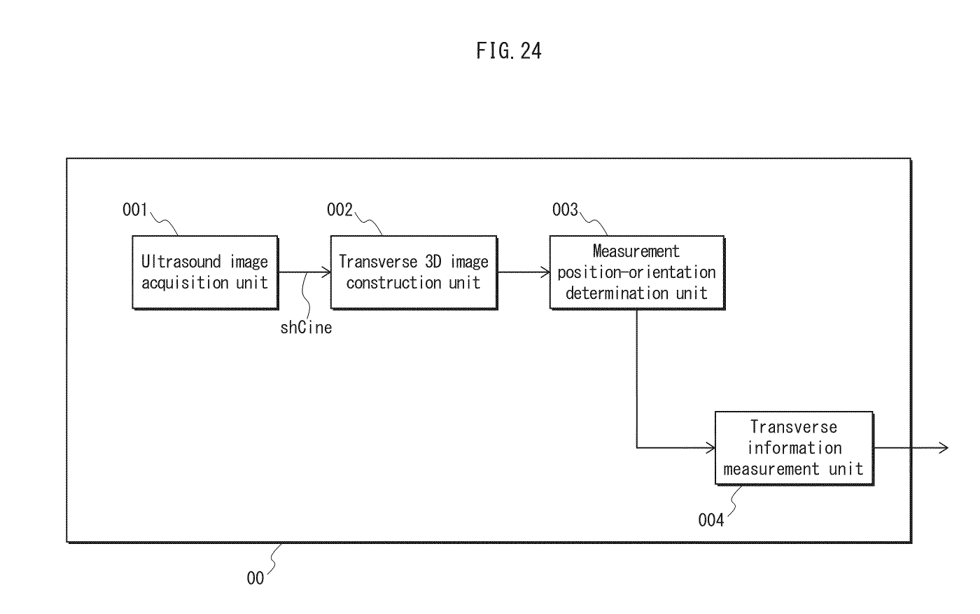

FIG. 24 is a block diagram illustrating configuration of an ultrasound diagnostic apparatus 00 envisaged by the inventors. The ultrasound diagnostic apparatus 00 includes an ultrasound image acquisition unit 001, a transverse 3D image construction unit 002, a measurement position-orientation determination unit 003 and a transverse information measurement unit 004. A 3D image of the carotid artery is constructed from a plurality of transverse tomographic images acquired by scanning along the carotid artery in the longitudinal direction using an ultrasound probe. Based on the 3D image, a predetermined position along a longitudinal cross-section is determined and IMT is calculated at a position in the 3D image corresponding to the position which is determined.

The ultrasound image acquisition unit 001 acquires a plurality of transverse tomographic images shCine through scanning along the carotid artery in the longitudinal direction and inputs the transverse tomographic images shCine into the transverse 3D image construction unit 002. The transverse 3D image construction unit 002 extracts contours of the carotid artery from each of the transverse tomographic images shCine and constructs a 3D image (referred to below as a transverse 3D image) by arranging the contours in a 3D space. The measurement position-orientation determination unit 003 determines a measurement position for IMT and an orientation of a maximum effective plane in a transverse cross-section, based on input by an operator. The transverse information measurement unit 004 measures IMT from a cross-section of the blood vessel in the transverse 3D image, which is defined by the measurement position and the maximum effective plane.

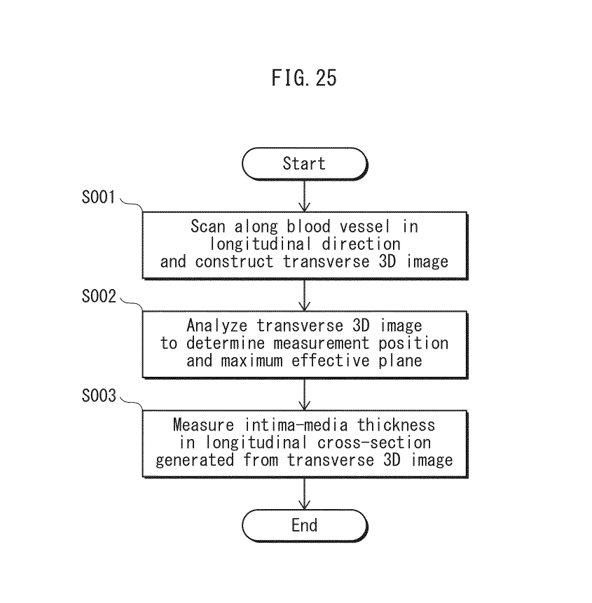

FIG. 25 is a flowchart illustrating operations of the ultrasound diagnostic apparatus 00 envisaged by the inventors. In Step S001 the carotid artery is scanned in the longitudinal direction and a transverse 3D image is constructed. In Step S002, the transverse 3D image is analyzed and based on input from an operator, a measurement position for IMT and a maximum effective plane is determined. In Step S003 IMT is measured from the transverse 3D image.

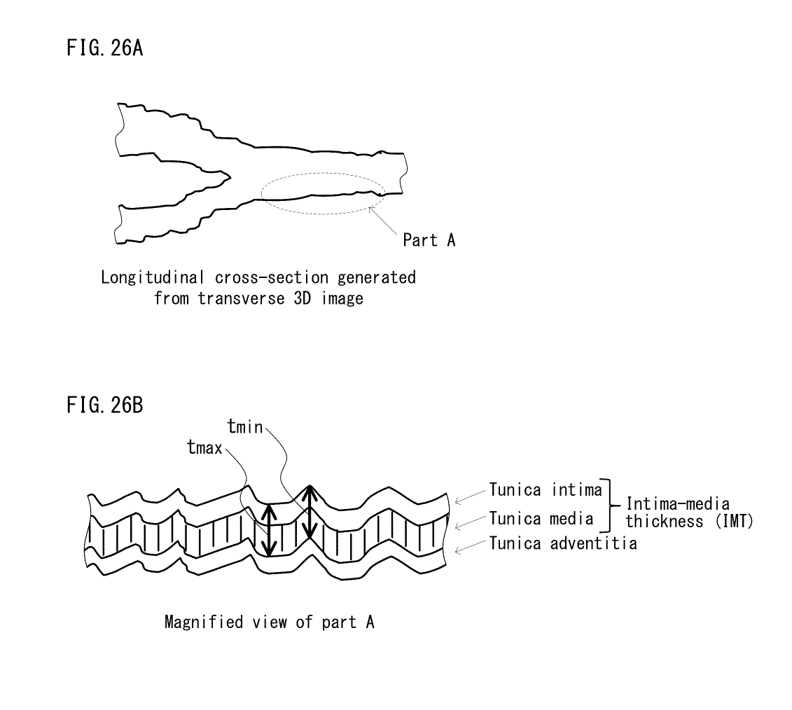

In the ultrasound diagnostic apparatus 00, IMT is measured based on the transverse 3D image, which is generated from the plurality of transverse tomographic images acquired through scanning the ultrasound probe along the carotid artery in the longitudinal direction. FIGS. 26A and 26B illustrate a longitudinal cross-section extracted from the transverse 3D image, which is generated from the plurality of transverse tomographic images acquired through scanning along the carotid artery in the longitudinal direction using the ultrasound image analysis device 00 envisaged by the inventors. A blood vessel pulsates in synchronization with beats of the heart, and therefore position and size of vascular contours of the blood vessel vary in accordance with the pulsations. When scanning along the carotid artery in the longitudinal direction, the ultrasound probe is moved in the longitudinal direction while scanning the entire length of the carotid artery. As a consequence of the above, each of the transverse tomographic images is acquired during a different phase of the pulsations. Hence, the vascular contours vary in size for transverse tomographic images acquired during different phases of the pulsations. Also, when a longitudinal cross-section is extracted from a transverse 3D image, which is generated using transverse tomographic images acquired during different phases of the pulsations, unevenness of the longitudinal cross-section arises such as illustrated in FIGS. 26A and 26B. Furthermore, vascular wall thickness in the longitudinal cross-section also varies in the longitudinal direction in accordance with the pulsations, thus variation occurs in results of IMT measurement performed using the longitudinal cross-section. As a consequence of the above variation, accurate IMT measurement may not be possible using the transverse tomographic images acquired by scanning along the carotid artery in the longitudinal direction.

As explained above, when measuring IMT of the vascular wall of the carotid artery, a region which is a predetermined distance from a measurement reference position can be defined as a target position in order to automatically determine a measurement target region for IMT. The measurement reference position is set based on external shape of the carotid artery. For example, the measurement reference position may be set based on a CCA-bulb boundary 219.

The inventors focused on an idea that once the measurement target region is determined from the transverse 3D image, if the ultrasound probe can be positioned in accordance with the measurement target region to newly acquire a longitudinal tomographic image, IMT can be measured without being affected by the pulsations. The inventors considered that in order to achieve the above, provision is required of a unit for detecting a position and an orientation of the ultrasound probe and a method for guiding the ultrasound probe to a measurement position and a measurement orientation for acquiring the longitudinal tomographic image of the measurement target region. Upon dedicated investigation into a method for determining a measurement target region for measuring IMT in a carotid artery and subsequently guiding, in a simple manner, an ultrasound probe to a position and an orientation for acquiring an ultrasound image of the measurement target region, the inventors conceived of an ultrasound diagnostic apparatus relating to each embodiment of the present disclosure.

The following explains, with reference to the drawings, an ultrasound diagnostic apparatus and a control method thereof relating to each of the embodiments.

### Outline of Embodiments

One non-limiting and exemplary embodiment provides an ultrasound diagnostic apparatus to which an ultrasound probe and a position-orientation detection unit, configured to detect a position and an orientation of the ultrasound probe, are connectable, the ultrasound diagnostic apparatus comprising: a transmission-reception processing unit configured to transmit an ultrasound wave toward a blood vessel which is a measurement target via the ultrasound probe and to receive a reflected ultrasound wave from the blood vessel via the ultrasound probe; a 2D image generation unit configured to generate a tomographic image of the blood vessel based on the reflected ultrasound wave; a measurement position-orientation determination unit configured to determine a measurement target region of the blood vessel, based on a 3D image of the blood vessel generated from a plurality of transverse tomographic images acquired by scanning of the ultrasound probe along the blood vessel in a longitudinal direction, and to determine a measurement position and a measurement orientation of the ultrasound probe for acquiring a longitudinal tomographic image including the measurement target region; a judgment unit configured to judge whether a current position and a current orientation of the ultrasound probe, detected by the position-orientation detection unit at a current time, differ from the measurement position and the measurement orientation respectively by no greater than a threshold value; and a property measurement unit configured to calculate a property of a vascular wall of the blood vessel in the measurement target region, wherein when the judgment unit judges that the current position and the current orientation differ from the measurement position and the measurement orientation respectively by no greater than the threshold value, the property measurement unit calculates the property of the vascular wall based on the longitudinal tomographic image of the blood vessel.

Alternatively, a display may be connectable to the ultrasound diagnostic apparatus, and the ultrasound diagnostic apparatus may further comprise a display control unit configured to control the display to display the 3D image of the blood vessel, the measurement position and the measurement orientation, and the current position and the current orientation of the ultrasound probe.

Alternatively, the ultrasound diagnostic apparatus may further comprise the position-orientation detection unit.

Alternatively, the ultrasound diagnostic apparatus may further comprise a transverse 3D image construction unit configured to construct the 3D image of the blood vessel based on the plurality of transverse tomographic images and based on position-orientation information indicating a position and an orientation of the ultrasound probe when each of the transverse tomographic images is acquired.

Alternatively, the blood vessel may be a carotid artery, and the property of the vascular wall may be an intima-media thickness.

Alternatively, the measurement position-orientation determination unit may determine the measurement target region for intima-media thickness based on a boundary position between a common carotid artery and a bulb of the common carotid artery, and may determine the measurement position and the measurement orientation in a manner such that the measurement target region is included in a reception signal acquisition range of the ultrasound probe when the ultrasound probe is positioned at the measurement position and the measurement orientation.

Alternatively, the measurement position-orientation determination unit may detect a position of maximum hypertrophy, at which the intima-media thickness is a maximum value, in at least one part of the carotid artery, and may determine the measurement position and the measurement orientation in a manner such that the position of maximum hypertrophy is included in a reception signal acquisition range of the ultrasound probe when the ultrasound probe is positioned at the measurement position and the measurement orientation, and the part of the carotid artery may be a common carotid artery, a bulb of the common carotid artery or an internal carotid artery.

Alternatively, the measurement position-orientation determination unit may detect a position of maximum hypertrophy, at which the intima-media thickness is a maximum value, in at least one part of the carotid artery, the part of the carotid artery may be a common carotid artery, a bulb of the common carotid artery or an internal carotid artery, and the property measurement unit may measure an intima-media volume in a region including the position of maximum hypertrophy, based on the 3D image of the blood vessel.

Another non-limiting and exemplary embodiment provides an ultrasound diagnostic apparatus to which an ultrasound probe and a position-orientation detection unit, configured to detect a position and an orientation of the ultrasound probe, are connectable, the ultrasound diagnostic apparatus comprising: a transmission-reception processing unit configured to transmit an ultrasound wave toward a blood vessel which is a measurement target via the ultrasound probe and to receive a reflected ultrasound wave from the blood vessel via the ultrasound probe; a 2D image generation unit configured to generate a tomographic image of the blood vessel based on the reflected ultrasound wave; a measurement position-orientation determination unit configured to determine a measurement target region of the blood vessel, based on a plurality of transverse tomographic images acquired by scanning of the ultrasound probe along the blood vessel in a longitudinal direction and based on position-orientation information indicating a position and an orientation of the ultrasound probe when each of the transverse tomographic images is acquired, and to determine a measurement position and a measurement orientation of the ultrasound probe for acquiring a longitudinal tomographic image including the measurement target region; a judgment unit configured to judge whether a current position and a current orientation of the ultrasound probe, detected by the position-orientation detection unit at a current time, differ from the measurement position and the measurement orientation respectively by no greater than a threshold value; and a property measurement unit configured to calculate a property of a vascular wall of the blood vessel in the measurement target region, wherein when the judgment unit judges that the current position and the current orientation differ from the measurement position and the measurement orientation respectively by no greater than the threshold value: the transmission-reception unit transmits the ultrasound wave and receives the reflected ultrasound wave via the ultrasound probe which is positioned at the current position and the current orientation; the 2D image generation unit generates the longitudinal tomographic image of the blood vessel based on the reflected ultrasound wave; and the property measurement unit calculates the property of the vascular wall based on the longitudinal tomographic image of the blood vessel.

Another non-limiting and exemplary embodiment provides an ultrasound diagnostic apparatus to which an ultrasound probe and a position-orientation detection unit, configured to detect a position and an orientation of the ultrasound probe, are connectable, the ultrasound diagnostic apparatus comprising: a transmission-reception processing unit configured to transmit an ultrasound wave toward a blood vessel which is a measurement target via the ultrasound probe and to receive a reflected ultrasound wave from the blood vessel via the ultrasound probe; a 2D image generation unit configured to generate a tomographic image of the blood vessel based on the reflected ultrasound wave; a transverse 3D image construction unit configured to construct a 3D image of the blood vessel based on a plurality of transverse tomographic images acquired by scanning of the ultrasound probe along the blood vessel in a longitudinal direction and based on position-orientation information indicating a position and an orientation of the ultrasound probe when each of the transverse tomographic images is acquired; a transverse information analysis unit configured to determine, based on the 3D image of the blood vessel, a measurement position and a measurement orientation of the ultrasound probe for acquiring a longitudinal tomographic image for measuring a property of a vascular wall of the blood vessel; a judgment unit configured to judge whether a current position and a current orientation of the ultrasound probe, detected by the position-orientation detection unit at a current time, differ from the measurement position and the measurement orientation respectively by no greater than a threshold value; a longitudinal information analysis unit configured to determine an updated measurement position of the ultrasound probe for acquiring a longitudinal tomographic image including a measurement target region of the blood vessel; a measurement position determination unit configured to determine the measurement target region of the blood vessel for measuring the property of the vascular wall, based on the updated measurement position; and a property measurement unit configured to calculate the property of the vascular wall in the measurement target region based on the longitudinal tomographic image, wherein when the judgment unit judges that the current position and the current orientation differ from the measurement position and the measurement orientation respectively by no greater than the threshold value: the transmission-reception unit transmits the ultrasound wave and receives the reflected ultrasound wave via the ultrasound probe which is located at the current position and the current orientation; the 2D image generation unit generates the longitudinal tomographic image of the blood vessel based on the reflected ultrasound wave; the longitudinal information analysis unit determines the updated measurement position based on the longitudinal tomographic image; the measurement position determination unit determines the measurement target region, for measuring the property of the vascular wall, based on the updated measurement position; and the property measurement unit calculates the property of the vascular wall in the measurement target region based on the longitudinal tomographic image of the blood vessel.

Another non-limiting and exemplary embodiment provides an ultrasound diagnostic apparatus to which an ultrasound probe is connectable which is configured such that at least one transducer column thereof, having a plurality of ultrasound transducers arranged in a column, is configured to scan in a row direction perpendicular to the column, the ultrasound diagnostic apparatus comprising: a transmission-reception processing unit configured to transmit an ultrasound wave toward a blood vessel which is a measurement target via the ultrasound probe and to receive a reflected ultrasound wave from the blood vessel via the ultrasound probe; a 2D image generation unit configured to generate a tomographic image of the blood vessel based on the reflected ultrasound wave; a measurement position-orientation determination unit configured to determine a measurement target region of the blood vessel for measuring a property of a vascular wall of the blood vessel based on a 3D image of vascular contours formed by arranging in a 3D space, vascular contours of the blood vessel extracted from a plurality of tomographic images generated by the 2D image generation unit, the tomographic images being acquired by scanning of the transducer column in the row direction along one direction of the blood vessel and the vascular contours being arranged based on each of the tomographic images and a row direction position of the transducer column when the tomographic image is acquired; a scan plane setting unit configured to determine a column position of the ultrasound transducers for acquiring a tomographic image that is parallel to the row direction and that includes the measurement target region, and to instruct the transmission-reception processing unit to perform transmission processing and reception processing for acquiring a tomographic image for property measurement at the column position; and a property measurement unit configured to measure the property of the vascular wall by analyzing the tomographic image for property measurement.

Alternatively, the ultrasound probe may be configured in a manner such that a plurality of transducer columns thereof, each having by a plurality of ultrasound transducers arranged in a column, are arranged in the row direction perpendicular to the column.

Alternatively, the ultrasound probe may be configured in a manner such that a transducer column thereof, having a plurality of ultrasound transducers arranged in a column, is configured to move in a direction perpendicular to the column.

Another non-limiting and exemplary embodiment provides a control method for an ultrasound diagnostic apparatus to which an ultrasound probe and a position-orientation detection unit, configured to detect a position and an orientation of the ultrasound probe, are connectable, the control method comprising: a step of transmitting an ultrasound wave toward a blood vessel which is a measurement target via the ultrasound probe and receiving a reflected ultrasound wave from the blood vessel via the ultrasound probe; a step of generating a tomographic image of the blood vessel based on the reflected ultrasound wave; a step of determining a measurement target region of the blood vessel based on a 3D image of the blood vessel generated from a plurality of transverse tomographic images acquired by scanning of the ultrasound probe along the blood vessel in a longitudinal direction, and determining a measurement position and a measurement orientation of the ultrasound probe for acquiring a longitudinal tomographic image including the measurement target region; a step of judging whether a current position and a current orientation of the ultrasound probe, detected by the position-orientation detection unit at a current time, differ from the measurement position and the measurement orientation respectively by no greater than a threshold value; and a step of calculating a property of a vascular wall of the blood vessel in the measurement target region, based on the longitudinal tomographic image, when the current position and the current orientation of the ultrasound probe differ from the measurement position and the measurement orientation respectively by no greater than the threshold value.

### First Embodiment

The following explains, with reference to the drawings, an ultrasound diagnostic apparatus relating to a first embodiment.

When an ultrasound diagnostic apparatus 10 relating to the first embodiment measures a property of a vascular wall of a blood vessel which is a measurement target, the ultrasound diagnostic apparatus 10 determines a measurement target region in the blood vessel for measuring the property of the vascular wall. The ultrasound diagnostic apparatus 10 automatically determines a measurement position and a measurement orientation for an ultrasound probe at which an ultrasound image can be acquired of a scan plane that includes the measurement target region. Herein, the term “scan plane” refers to a region of which an ultrasound image can be acquired. In addition to the above, the ultrasound diagnostic apparatus 10 guides an operator using a display screen in a manner such that the ultrasound image can be acquired of the scan plane indicated by the measurement position and the measurement orientation. The ultrasound diagnostic apparatus 10 also automatically judges whether a position and an orientation of the ultrasound probe operated by the operator match the measurement position and the measurement orientation respectively. When the above positions and orientations match, the ultrasound diagnostic apparatus 10 acquires an ultrasound image thereat and measures the property of the vascular wall from the ultrasound image. The present embodiment is explained using IMT of a carotid artery as an example of the property of the vascular wall of the blood vessel, which is the measurement target.

<Configuration>

(General Configuration)

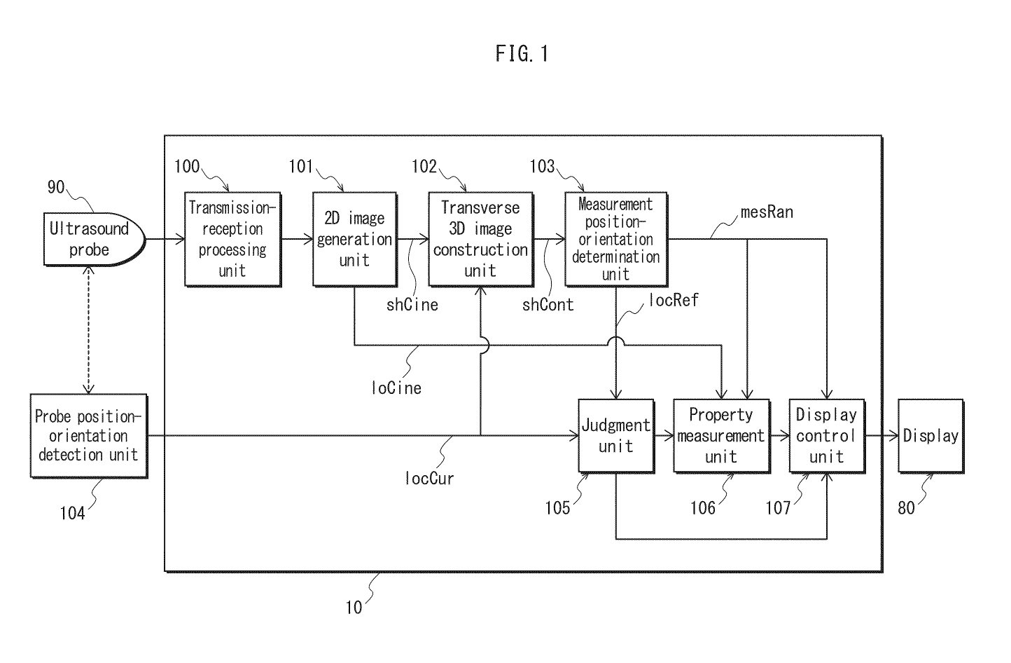

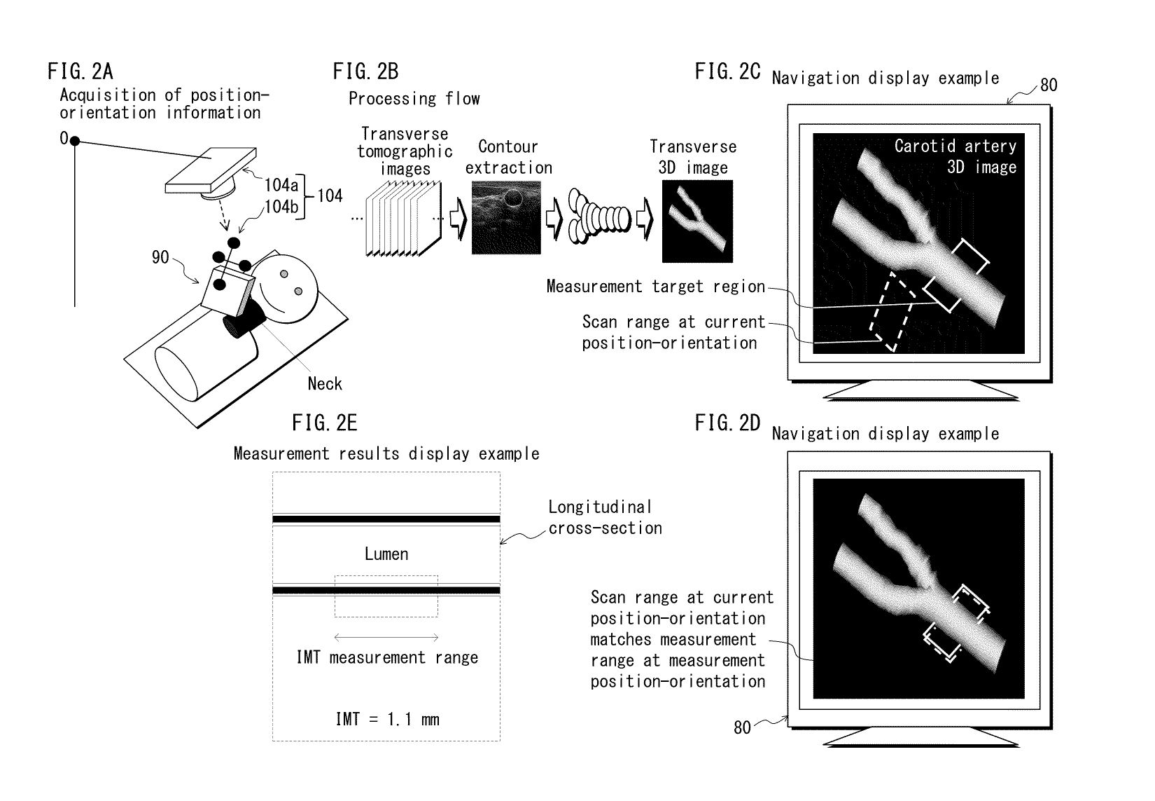

FIG. 1 is a block diagram illustrating functional configuration of the ultrasound diagnostic apparatus 10 relating to the first embodiment. FIGS. 2A-2E illustrate an overview of functions of the ultrasound diagnostic apparatus 10.

As illustrated in FIG. 1, the ultrasound diagnostic apparatus 10 is configured in a manner such that an ultrasound probe 90, a probe position-orientation detection unit 104 and a display 80 are each electrically connectable to the ultrasound diagnostic apparatus 10. The ultrasound probe 90 transmits an ultrasound wave towards a subject and receives a reflected ultrasound wave from the subject, the probe position-orientation detection unit 104 detects a position and an orientation of the ultrasound probe 90, and the display 80 displays information. FIG. 1 illustrates the ultrasound diagnostic apparatus 10 with the ultrasound probe 90, the probe position-orientation detection unit 104 and the display 80 each connected thereto. The ultrasound diagnostic apparatus 10 includes a transmission-reception processing unit 100, a 2D image generation unit 101, a transverse 3D image construction unit 102, a measurement position-orientation determination unit 103, a judgment unit 105, a property measurement unit 106 and a display control unit 107.

(Ultrasound Probe 90)

The ultrasound probe 90 includes a transducer column, which has a plurality of piezoelectric elements arranged in a column as a 1D array (not illustrated). The ultrasound probe 90 receives a transmission signal supplied from the transmission-reception processing unit 100 as a pulse or continuous electrical signal, and converts the transmission signal into a pulse or continuous ultrasound wave. While the transducer column is in contact with skin surface of the subject, the ultrasound probe 90 emits an ultrasound beam from the skin surface toward the carotid artery. In order to acquire a tomographic image which is a transverse cross-section of the carotid artery, the ultrasound beam is emitted while the ultrasound probe 90 is orientated such that the transducer column is perpendicular to the longitudinal direction of the carotid artery. The ultrasound probe 90 also receives an ultrasound echo signal which is an ultrasound wave reflected from the subject. The ultrasound probe 90 converts the ultrasound echo signal into an electrical signal through the transducer column, and supplies the electrical signal to the transmission-reception processing unit 100.

In order that a plurality of transverse tomographic images of the carotid artery can be acquired, the ultrasound probe 90 is scanned along the carotid artery in the longitudinal direction with the ultrasound probe 90 orientated such that the transducer column is approximately perpendicular to the longitudinal direction of the carotid artery. The above operation is referred to below as a “hand scan”. FIG. 2A is a schematic diagram of a situation in which the ultrasound probe 90 is used to perform a hand scan along the carotid artery in the longitudinal direction. The transducer column of the ultrasound probe 90 is applied against the skin surface and an ultrasound beam is transmitted while moving the ultrasound probe 90 in a single direction longitudinally along the carotid artery. During the above, the ultrasound probe 90 should preferably be moved along the carotid artery in the longitudinal direction at a constant speed in order that the plurality of transverse tomographic images can be acquired at constant intervals relative to one another.

For example, when a degree of allowable error of a measurement plane when acquiring a transverse tomographic image is 0.25 mm and when a frame acquisition rate is 20 frames/sec, scanning should preferably be performed in the longitudinal direction at a speed of 5 mm/sec.

Through the above, the ultrasound probe 90 receives an ultrasound echo signal for a transverse cross-section of the carotid artery corresponding to a position which the ultrasound probe 90 is moved to. Subsequently, the ultrasound probe 90 converts the ultrasound echo signal to an electrical signal and supplies the electrical signal to the transmission-reception processing unit 100.

(Probe Position-Orientation Detection Unit 104)

The probe position-orientation detection unit 104 detects a position and an orientation of the ultrasound probe 90, and outputs the position and the orientation to the transverse 3D image construction unit 102 and the judgment unit 105, which are explained further below. As illustrated in FIG. 2A, the probe position-orientation detection unit 104 is for example configured by an image capture sub-unit 104a, such as a CCD camera, and optical markers 104b which are attached to the ultrasound probe 90, for example at four different locations. The optical markers 104b are captured by the image capture sub-unit 104a and based on positions of the optical markers 104b, relative positions of the optical markers 104b relative to one another, and change in the positions and the relative positions, the probe position-orientation detection unit 104 detects in real time, the position and the orientation of the ultrasound probe 90 in 3D space. In the above, the subject is assumed not to move during examination of IMT and the operator is assumed to possess enough procedural skill to be able to perform a hand scan of the ultrasound probe 90 along the carotid artery of the subject in the longitudinal direction, in order to acquire transverse tomographic images of the carotid artery. When the operator performs the hand scan to acquire the plurality of transverse tomographic images of the carotid artery, with regards to each of the transverse tomographic images, a position and an orientation of the ultrasound probe 90 is detected for a point in time when the transverse tomographic image is acquired. The probe position-orientation detection unit 104 sends the position and the orientation of the ultrasound probe 90 when each of the transverse tomographic images is acquired to the transverse 3D image construction unit 102 and the judgment unit 105, in accompaniment to an order in which the transverse tomographic image is acquired during the hand scan. The transverse 3D image construction unit 102 and the judgment unit 105 are explained further below.

FIG. 2A illustrates one example of the probe position-orientation detection unit 104 detecting the position and the orientation of the ultrasound probe 90. In order that an image of the optical markers 104b can be acquired using the image capture sub-unit 104a, the image capture sub-unit 104a is positioned so that the optical markers 104b are not positioned in a blind spot of the image capture sub-unit 104a, even when the ultrasound probe 90 is moved. Herein, an example is given in which the image capture sub-unit 104a is positioned above the subject in order to reduce blind spot occurrence. Furthermore, by positioning a plurality of image capture sub-units 104a, blind spot occurrence can be further reduced due to each of the optical markers 104b only needing to be visible from one of the image capture sub-units 104a. Thus, the position and the orientation of the ultrasound probe 90 can be detected while the ultrasound probe 90 is scanned along the carotid artery in the longitudinal direction.

(Transmission-Reception Processing Unit 100)

The transmission-reception processing unit 100 performs transmission processing to generate a pulse or continuous electrical signal for causing the ultrasound probe 90 to transmit an ultrasound beam and to supply the electrical signal to the ultrasound probe 90 as a transmission signal.

The transmission-reception processing unit 100 also performs reception processing to amplify and A/D convert an electrical signal received from the ultrasound probe 90, thus generating a reception signal. The reception signal is for example formed from a plurality of signals in a direction along the transducer column and in a depth direction away from the transducer column Each of the signals is an A/D converted digital signal of an electrical signal converted from amplitude of an echo signal. Through the above, a reception signal is generated for a transverse cross-section of the carotid artery in each of a plurality of frames in the hand scan described above. The transmission-reception processing unit 100 supplies the reception signal for each of the plurality of frames to the 2D image generation unit 101.

(2D Image Generation Unit 101)

Based on each of the reception signals, the 2D image generation unit 101 generates a 2D image shCine which is a transverse tomographic image of the carotid artery for a frame to which the reception signal corresponds. Thus, the 2D image generation unit 101 generates a plurality of 2D images shCine. The 2D image generation unit 101 supplies the 2D images shCine to the transverse 3D image construction unit 102. Each of the 2D images shCine is an image signal, the image signal being the reception signal on which coordinate conversion has been performed so as to correspond to an orthogonal coordinate system. The 2D image generation unit 101 supplies each of the 2D images shCine to the transverse 3D image construction unit 102, accompanied by an order in which the 2D image shCine is acquired during the hand scan.

(Transverse 3D Image Construction Unit 102)

The transverse 3D image construction unit 102 extracts vascular contours of the carotid artery from each of the 2D images shCine. As illustrated in FIG. 2B, extraction of vascular contours is performed based on the transverse tomographic images, for example using a common image processing technique, such as edge detection processing, in order to extract contours of parts of the vascular wall of the blood vessel. Subsequently, the vascular contours from each of the transverse tomographic images are mapped in a 3D space to construct a 3D image of the carotid artery based on position and orientation of a scan plane, which is calculated from the position and the orientation of the ultrasound probe 90 at which the transverse tomographic image is acquired, received from the probe position-orientation detection unit 104. The 3D image of the carotid artery is for example constructed by generating a polygon from contour apices of each of the transverse tomographic images. The transverse 3D image construction unit 102 maps the vascular contours in the 3D space and outputs coordinates shCont of the vascular contours to the measurement position-orientation determination unit 103.

(Measurement Position-Orientation Determination Unit 103)

Based on the vascular contours and the coordinates shCont of the vascular contours, the measurement position-orientation determination unit 103 analyzes 3D shape of the vascular contours. The measurement position-orientation determination unit 103 determines measurement position-orientation information locRef indicating a position and an orientation of the ultrasound probe 90 configuring a scan plane at which a longitudinal tomographic image for IMT measurement can be acquired. The measurement position-orientation determination unit 103 also determines measurement range information mesRan indicating a measurement range for IMT.

For example, based on the vascular contours and the coordinates shCont thereof, the measurement position-orientation determination unit 103 calculates variation in outer diameter of the tunica adventitia in the longitudinal direction and detects a point of inflection as a boundary position 219 (herein, referred to as a CCA-bulb boundary 219) between the CCA 213 and the bulb 214. Using the CCA-bulb boundary 219 as a point of origin, the measurement position-orientation determination unit 103 determines an IMT measurement range 212 to be a range extending for 1-2 cm from a start point, which is a point 1 cm toward the CCA 213 from the point of origin. The measurement position-orientation determination unit 103 outputs the IMT measurement range 212 which is determined as measurement range information mesRan. The measurement position-orientation determination unit 103 also calculates a measurement position and a measurement orientation, which are respectively a position and an orientation of the ultrasound probe 90 at which a longitudinal tomographic image of a scan plane including the IMT measurement range 212 can be acquired. The measurement position-orientation determination unit 103 outputs the measurement position and the measurement orientation as measurement position-orientation information locRef.

The measurement position and the measurement orientation are not limited to being determined by the method described above. For example, alternatively position of the tunica adventitia or variation of IMT in the longitudinal direction may be calculated, a point of inflection thereof may be detected as the CCA-bulb boundary 219, and the IMT measurement range 212 may be defined as a predetermined range relative to a point of origin which is set as the CCA-bulb boundary 219 which is detected.

IMT measurement should preferably be performed using a maximum effective plane which passes centrally through the carotid artery. The above is due to an ultrasound signal being incident approximately perpendicular to a near wall and a far wall of vascular contours in a transverse tomographic image of the maximum effective plane, and thus a stronger reflected wave can be acquired by the ultrasound transducers.

The measurement position-orientation determination unit 103 outputs the measurement position-orientation information locRef and the measurement range information mesRan to the display control unit 107. Subsequently, navigation is performed in order to guide the operator to move the ultrasound probe 90 to the measurement position and the measurement orientation, which indicate position and orientation of a scan plane.

(Judgment Unit 105)

The judgment unit 105 receives current position-orientation information locCur from the probe position-orientation detection unit 104, which indicates a current position and a current orientation of the ultrasound probe 90 being operated by the operator.

The judgment unit 105 performs a comparison of the measurement position-orientation information locRef and the position-orientation information locCur to judge whether the current position-orientation information locCur differs from the measurement position-orientation information locRef by no greater than a predetermined threshold value. The current position-orientation information locCur, indicating the current position and the current orientation of the ultrasound probe 90, is acquired directly from the probe position-orientation detection unit 104. On the other hand, the measurement position-orientation information locRef is information indicating a position and an orientation of the ultrasound probe 90 which configure a scan plane at which a longitudinal tomographic image for IMT measurement can be acquired. The judgment unit 105 is able to directly compare the two types of information described above.

In a situation in which the current position-orientation information locCur indicates the current position and the current orientation of the ultrasound probe 90, and the measurement position-orientation information locRef for example indicates a position and an orientation of a scan plane at which the longitudinal tomographic image for IMT can be acquired, the two types of information cannot be directly compared. In the above situation, in order that the two types of information can be directly compared, the measurement position-orientation information locRef is required to be converted into a position and an orientation of the ultrasound probe 90 which configure the scan plane described above.

Alternatively, the two types of information can be directly compared in a situation in which the current position-orientation information locCur indicates a position and an orientation of a scan plane currently configured by the ultrasound probe 90 and the measurement position-orientation information locRef indicates the position and the orientation of the scan plane at which the longitudinal tomographic image for IMT measurement can be acquired.

The judgment unit 105 outputs the current position-orientation information locCur to the display control unit 107, indicating the current position and the current orientation of the ultrasound probe 90.

When the current position-orientation information locCur differs from the measurement position-orientation information locRef by no greater than the predetermined threshold value, the judgment unit 105 may instruct the transmission-reception processing unit 100 to perform transmission processing and reception processing to acquire a longitudinal tomographic image for property measurement, thus newly acquiring a longitudinal tomographic image. The 2D image generation unit 101 receives a reception signal from the transmission-reception processing unit 100, generates a 2D image loCine which is a longitudinal tomographic image based on the reception signal and supplies the 2D image loCine to the property measurement unit 106, which is explained further below.

(Property Measurement Unit 106)

The property measurement unit 106 acquires the 2D image loCine from the 2D image generation unit 101 and measures IMT in a range indicated by the measurement range information mesRan.

Through the above, the property measurement unit 106 measures IMT based on the 2D image loCine, which is a longitudinal tomographic image acquired at the current position when the judgment unit 105 judges that the current position-orientation information locCur differs from the measurement position-orientation information locRef by no greater than the threshold value.

As explained above, a vascular wall is configured by a tunica intima, a tunica media and a tunica adventitia in order of innermost position and IMT refers to intima-media thickness which is thickness of an intima-media complex. The property measurement unit 106 measures IMT by detecting the intima-media between the lumen and the tunica adventitia in a 2D image generated based on a reception signal. IMT is measured from the longitudinal tomographic image of the blood vessel using a method which is, for example, based on a method recited in International Publication No. WO2007/108359. The display 80 is controlled to display results of IMT measurement.

(Display Control Unit 107)

The display control unit 107 receives, from the measurement position-orientation determination unit 103, the measurement range information mesRan, which indicates the IMT measurement range, and the measurement position-orientation information locRef, which indicates the measurement position and the measurement orientation of the ultrasound probe 90 configuring the scan plane in the transverse 3D image at which the longitudinal tomographic image for IMT measurement can be acquired. The display control unit 107 controls the display 80 to display the scan plane and the IMT measurement range superimposed on the transverse 3D image.

The display control unit 107 also receives, from the judgment unit 105, the current position-orientation information locCur, which indicates the current position and the current orientation of the ultrasound probe 90 being operated by the operator. The display control unit 107 controls the display 80 to display a scan plane configured by the ultrasound probe 90 at the current position and the current orientation indicated by the current position-orientation information locCur.

The display control unit 107 also receives, from the property measurement unit 106, information indicating results of IMT measurement and controls the display 80 to display the results of IMT measurement. During the above, a more user friendly configuration is achieved by displaying the IMT measurement range 212, for which IMT measurement is performed, in accompaniment with the 3D image.

FIG. 2C illustrates an example of navigation display presented to the operator. A scan plane (solid-line rectangle) including a measurement target region set by the measurement position-orientation information locRef and a current scan plane (dashed-line rectangle) calculated from the current position and the current orientation of the ultrasound probe 90 are displayed superimposed on the 3D image of the carotid artery. While viewing the navigation display, the operator should preferably move the ultrasound probe 90 in order that position of the dashed-line rectangle matches position of the solid-line rectangle. When at a position such as illustrated in FIG. 2D, where difference between the two rectangles is no greater than a threshold value, a longitudinal tomographic image is acquired at the position. IMT measurement is performed based on the longitudinal tomographic image which is acquired and the IMT measurement range indicated by the measurement range information mesRan, and results of the IMT measurement are displayed as illustrated in FIG. 2E.

<Operation>

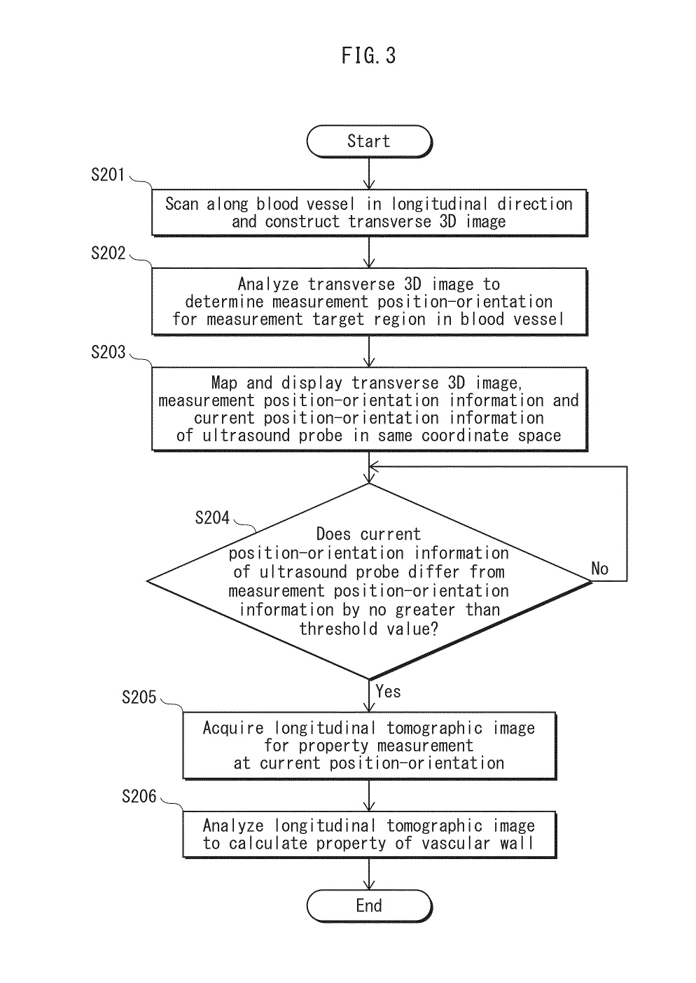

Operation of the ultrasound diagnostic apparatus 10, configured as described above, is explained below with reference to FIG. 3. FIG. 3 is a flowchart illustrating operations related to IMT measurement in the ultrasound diagnostic apparatus 10 relating to the first embodiment. Transmission of an ultrasound beam toward a carotid artery of a subject and reception of an ultrasound beam from the carotid artery is achieved using a conventional method, and therefore explanation thereof is omitted. In other words, the following explains operations up until automatic determination of IMT measurement range and performance of IMT measurement in the IMT measurement range.

(Step S201)

In Step S201, a hand scan of the ultrasound probe 90 along the carotid artery in the longitudinal direction is performed with the transducer column of the ultrasound probe 90 orientated approximately perpendicularly to the longitudinal direction, thus acquiring a plurality of transverse tomographic images of the carotid artery. Subsequently, vascular contours of the carotid artery are extracted from each of the transverse tomographic images. A 3D image of the carotid artery is constructed by mapping the vascular contours extracted from each of the transverse tomographic images to a 3D space, based on a position and an orientation of a scan plane corresponding to the transverse tomographic image. During the above, the position and the orientation of the scan plane is calculated from a position and an orientation of the ultrasound probe 90 when the transverse tomographic image corresponding to the scan plane is acquired. The position and the orientation of the ultrasound probe 90 are received from the probe position-orientation detection unit 104. Among the tunica intima, the tunica media and the tunica adventitia, contours are extracted of at least the tunica adventitia.

(Step S202)

In Step S202, shape of contours of the tunica adventitia in the transverse 3D image is analyzed to determine a measurement position and a measurement orientation of the ultrasound probe 90, at which a tomographic image can be acquired including a measurement target region of the blood vessel. In order that IMT measurement can be performed using a longitudinal tomographic image, the measurement position and the measurement orientation are determined by determining a position and an orientation of the ultrasound probe 90 configuring a scan plane for acquiring a longitudinal tomographic image including the measurement target region.

For example, as explained above, based on the vascular contours and the coordinates shCont thereof, variation in outer diameter of the tunica adventitia in the longitudinal direction is calculated and a point of inflection is detected as the CCA-bulb boundary 219. The CCA-bulb boundary 219 is taken as a point of origin and a point 1 cm toward the CCA 213 from the point of origin is taken as a start point of the IMT measurement range 212, which is a range of predetermined size starting therefrom. Subsequently, a measurement position and a measurement orientation of the ultrasound probe 90 are calculated for acquiring a longitudinal tomographic image of a scan plane which includes the IMT measurement range 212. The measurement position and the measurement orientation are output as measurement position-orientation information locRef. IMT measurement should preferably be performed using a maximum effective plane which passes centrally through the carotid artery and which has a predetermined orientation in the transverse direction.

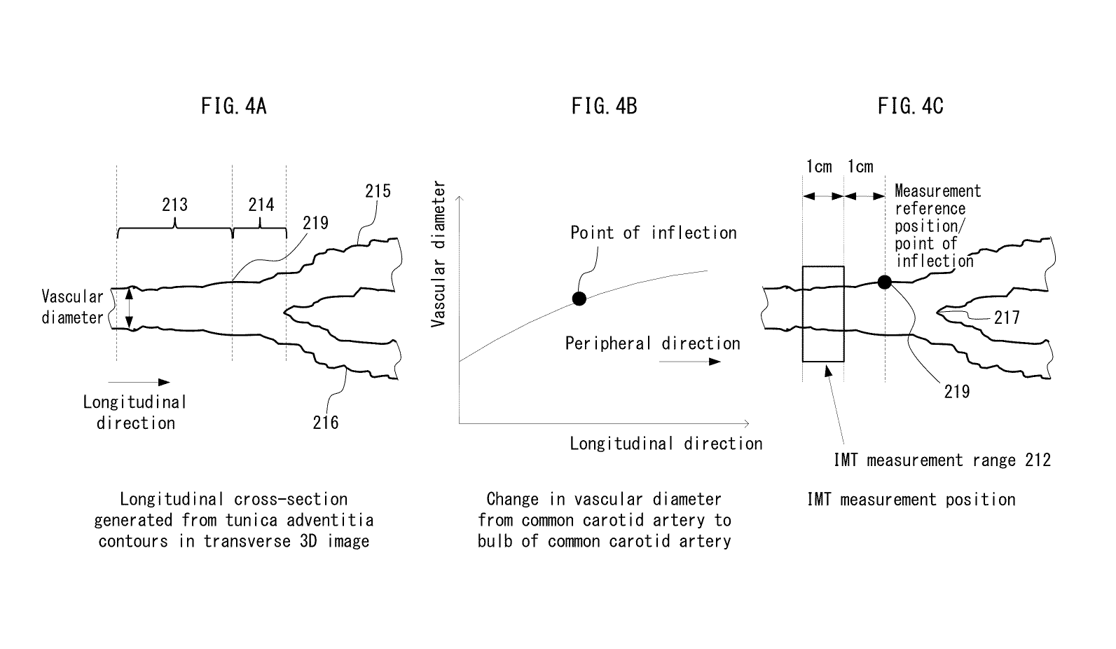

FIGS. 4A-4C are diagrams for explaining operations of the ultrasound diagnostic apparatus 10, relating to the first embodiment, in Step S202. FIG. 4A illustrates a longitudinal cross-section view of the contours of the tunica adventitia in the transverse 3D image constructed in Step S201. In early stage arteriosclerosis, hypertrophy of the intima-media complex close to the bulb 214 is common. Therefore, in screening for arteriosclerosis IMT measurement is recommended in a range extending for 1-2 cm from a start point, which is set as a point 1 cm toward the CCA 213 from the CCA-bulb boundary 219. The CCA-bulb boundary 219 can be detected as a point of inflection in a curve of vascular diameter.

FIG. 4B is a graph illustrating a curve of vascular diameter plotted on the vertical axis against distance in the longitudinal direction plotted on the horizontal axis for when the carotid artery is scanned in the longitudinal direction from the central end to the peripheral end thereof. The point of inflection is a position at which curvature of the curve of vascular diameter changes from positive to negative. Herein, the point of inflection is determined based on change in diameter in a longitudinal cross-section which is 1D information, however alternatively 2D information may be used such as change in area of transverse contours in the direction of the scan. By using change in area, location of the point of inflection can be determined in a manner which is less affected by errors in contour extraction, which for example occur due to fluctuation in contour position. Alternatively, the point of inflection may be calculated after performing low-pass filter processing on the vascular contours with regards to the longitudinal direction in order to reduce noise.

FIG. 4C illustrates the measurement target region as a region of 1 cm in width extending from a position 1 cm toward the CCA 213 from the point of inflection, which indicates the CCA-bulb boundary 219. Herein, the point of inflection is one example of a measurement reference position for determining the measurement target region. Alternatively, a different position such as the Bif 217 may be used as the measurement reference position. Furthermore, distance from the measurement reference point, measurement range and the like are defined differently in different diagnostic protocols and therefore are not limited to being 1 cm such as described above.

The longitudinal cross-section illustrated in FIG. 4A is a flat plane passing through the CCA 213 or the bulb 214 and also passing through a central position of vascular contours of each of the ICA 215 and the ECA 216. However, the longitudinal tomographic image may alternatively be an arbitrary flat plane including a central line of vascular contours of each of the CCA 213 and the bulb 214.

The maximum effective plane is selected as a flat plane which includes a central line of a part of the carotid artery close to the measurement reference position. Preferably the maximum effective plane should be determined using a preset method which has a high degree of reproducibility. For example, the maximum effective plane may be determined to be a flat plane including the central line close to the measurement reference position and also a central line of contours of the ICA 215 close to the Bif 217. Alternatively, the maximum effective plane may be determined to be a flat plane of least squares in terms of distance from the central line close to the measurement reference position and also a central line of contours of each of the ICA 215 and the ECA 216 close to the Bif 217. Further alternatively, the maximum effective plane may be determined and subsequently the measurement reference position may be determined based on contours of the tunica adventitia in the maximum effective plane.

A plurality of measurement target regions may alternatively be set, for example by setting a plurality of measurement target regions in the CCA 213 or by setting measurement target regions in a plurality of different parts of the carotid artery such as the CCA 213 and the ICA 215. The maximum effective plane may also be set as a plurality of different cross-sections, for example maximum effective planes may be set at three different orientations, each separated by an interval of 60 degrees. The CCA-bulb boundary 219 may alternatively be set as a position at which a gradient of the curve illustrated in FIG. 4B exceeds a threshold value, or as a position at which the vascular diameter or vascular area increases to a predetermined value relative to the CCA 213.

(Step S203)

In Step S203, based on the measurement position-orientation information locRef and the current position-orientation information locCur, a current scan plane and a target scan plane are mapped in the same coordinate space and presented to the operator as navigation information displayed on the display 80. The operator moves the ultrasound probe 90 in accordance with the navigation information. Once the ultrasound probe 90 is moved to the measurement position and measurement orientation by the operator, a longitudinal tomographic image is acquired.

The current scan plane, which is a scan plane of the ultrasound probe 90 at the current position and the current orientation, and the target scan plane, which is a scan plane of the ultrasound probe 90 when at the measurement position and the measurement orientation, are displayed on a navigation screen in a manner such that relative positions thereof can be easily understood.

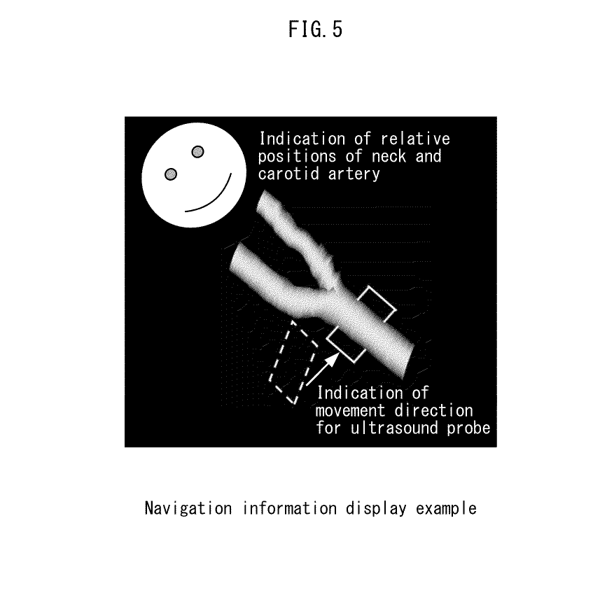

FIG. 5 illustrates a display example of navigation information in the ultrasound diagnostic apparatus 10 relating to the first embodiment. As illustrated in FIG. 5, the 3D image of the carotid artery and position of a head of the subject, or the target scan plane and the current scan plane, may be displayed with guidance information, such as a direction in which the ultrasound probe 90 should be moved.

(Step S204)

In Step S204 a judgment is performed as to whether the current position-orientation information locCur differs from the measurement position-orientation information locRef by no greater than the threshold value, and when the judgment is affirmative, operations proceed to Step S205. When the judgment is negative, the operator continues to move the ultrasound probe 90 until the current position-orientation locCur differs from the measurement position-orientation information locRef by no greater than the threshold value.

(Step S205)

In Step S205, a longitudinal tomographic image is acquired at the current position of the ultrasound probe 90.

(Step S206)

In Step S206, IMT measurement is performed in the measurement range indicated by the measurement range information mesRan, based on the ultrasound image acquired in Step S205. IMT measurement is performed by detecting the lumen-intima interface and the media-adventitia interface. A sudden change in brightness values in a B mode image is observed at the above interfaces, thus when a tomographic image is scanned perpendicular to the interfaces, the interfaces can be detected based on change in brightness values. A specific limitation on blood vessel shape, such as smooth change in blood vessel shape, may also be used as complementary information when detecting the interfaces. Also, IMT measurement is not limited to measurement of an average value of IMT in the measurement range, and alternatively a maximum value of IMT in the measurement range may be measured.

<Effects>

As explained above, through the ultrasound diagnostic apparatus 10, the operator is presented with a measurement position and a measurement orientation of the ultrasound probe 90 during navigation, which configure a scan plan at which a longitudinal tomographic image can be acquired of a measurement target region for IMT in a blood vessel. Therefore, the operator is always able to acquire a longitudinal tomographic image including the measurement target region, and thus is able to accurately measure IMT.

Also, a longitudinal tomographic image is newly acquired at the measurement position and the measurement orientation for acquiring the longitudinal tomographic of measurement target region, and IMT measurement is performed based on the longitudinal tomographic image which is acquired. Therefore, a problem does not occur of results of IMT measurement varying in the longitudinal direction in accordance with vascular pulsations, such as occurs when IMT measurement is performed based on a longitudinal cross-section generated from a transverse 3D image.

Furthermore, the operator is only required to move the ultrasound probe 90 to the measurement position and the measurement orientation which are presented to the operator, thus IMT measurement can be easily performed even without a high degree of procedural skill.

### Modified Examples

The above is an explanation of the ultrasound diagnostic apparatus 10 relating to the present embodiment. The ultrasound diagnostic apparatus in the present disclosure is of course not limited to the ultrasound diagnostic apparatus explained in the present embodiment and various modified examples of the ultrasound diagnostic apparatus in the present embodiment are possible, such as explained below.

(1) Explanation of the ultrasound diagnostic apparatus 10 uses the carotid artery as an example of a measurement target, however the measurement target is not limited to the carotid artery. Alternatively, the measurement target may be a different blood vessel such as an abdominal aorta or a tibial artery, or may be a different body part such as a breast or liver. Also, so long as surface of the body is scanned while an operator moves a probe, an image may be acquired using an image modeling diagnostic device other than the ultrasound probe, for example a device which uses photo-acoustic ultrasound waves or near infra-red light.

(2) For the ultrasound diagnostic apparatus 10, the probe position-orientation detection unit 104, which is connectable to the ultrasound diagnostic apparatus 10, is explained using an example in which the probe position-orientation detection unit 104 is configured by the image capture sub-unit 104a and the optical markers 104b. However, the probe position-orientation detection unit 104 is not limited to the above configuration. For example, the position and the orientation of the ultrasound probe may alternatively be detected using a magnetic sensor, an acceleration sensor or a gyroscope. If a magnetic sensor is used, the position and the orientation of the ultrasound probe can be detected by attaching a receiver of the magnetic sensor to the ultrasound probe and detecting change in a magnetic field generated by a magnetic field generator.

(3) For the ultrasound diagnostic apparatus 10, in Step S201 the ultrasound probe 90 is scanned in the longitudinal direction and a transverse 3D image is constructed. However, scan direction is not limited to the above. So long as an overall image of the carotid artery can be acquired over a range which includes the measurement target region, the scan may alternatively acquire a plurality of longitudinal tomographic images, or may acquire a combination of transverse and longitudinal tomographic images. Even when a scan is performed in a different direction, vascular contours can be extracted from ultrasound images which are acquired.

(4) The ultrasound diagnostic apparatus 10 has a configuration in which a phase is not stipulated for acquisition of the longitudinal tomographic image in Step S205. However, vascular diameter and IMT vary in accordance with vascular pulsations. Therefore, alternatively a configuration may be adopted in which measurement is performed in a phase corresponding to end-diastolic timing, during which vascular diameter is at a minimum value. In the above configuration, judgment is performed in Step S205 as to whether a current phase of vascular pulsations matches a predetermined phase, in addition to performing the judgment of difference between the two types of position information. The current phase of vascular pulsations may be acquired through an external device such as an electrocardiogram device or may be acquired through analysis of movement in ultrasound images. For example, by keeping the ultrasound probe stationary, for at least one heart beat, at a position where the two types of position information differ by no greater than the threshold value, a phase can be selected in which diameter or area of vascular contours is a minimum value. Alternatively, a plurality of ultrasound images may be acquired while the two types of position information differ by no greater than the threshold value. Further alternatively, IMT may be measured as an average value of IMT in a fixed period during end-diastolic timing of the heart beat.

(5) The ultrasound diagnostic apparatus 10 constructs a 3D image in Step S202, however so long as positions in a 3D space can be acquired for vascular contours extracted from ultrasound images, construction of a 3D image is not essential.

(6) The ultrasound diagnostic apparatus 10 has a configuration in which the transverse 3D image construction unit constructs a transverse 3D image from a plurality of transverse tomographic images generated through the transmission-reception processing unit 100 and the 2D image generation unit 101, based on tomographic images acquired by the ultrasound probe. However, a 3D image or a plurality of transverse tomographic images used to construct a transverse 3D image may be acquired through a different imaging modality to the ultrasound diagnostic apparatus, such as CT or MRI. In the above configuration, by calculating in advance a correspondence between a 3D coordinate system when the 3D image is acquired and the position and the orientation of the ultrasound probe, the ultrasound probe can be guided to a position and an orientation of a measurement target region determined from the 3D image.

(7) It is not essential that all processing in Steps 5202 and 5205 is performed automatically, and alternatively a portion of processing in Step S202 or a portion of processing in Step S205 may be performed manually by the operator.

(8) The ultrasound diagnostic apparatus 10 has a configuration in which navigation is performed with regards to a scan plane for acquiring a longitudinal tomographic image for IMT measurement. Alternatively, navigation may also be performed when acquiring a plurality of transverse tomographic images for constructing a transverse 3D image. For example, when scanning in the longitudinal direction to acquire the plurality of transverse tomographic images, information may be displayed on the navigation screen indicating whether movement speed is within a recommended range, in order that the ultrasound probe is moved at an optimal speed in accordance with a frame rate of ultrasound image acquisition. Furthermore, instead of constructing a transverse 3D image after completion of scanning to acquire transverse tomographic images of a region to be examined, alternatively transverse 3D images may be constructed one after another in real-time or close to real-time while scanning in the longitudinal direction, using transverse tomographic images of which acquisition is complete. Through the above configuration, scanning can be performed while checking a region of which scanning is already complete, thus facilitating judgment as to whether or not a required region is included in the scan.

(9) The ultrasound diagnostic apparatus 10 relating to the first embodiment has a configuration in which IMT is the property of the vascular wall of the carotid artery which is measured. However, the present disclosure is not limited by the above and in an alternative configuration the property of the vascular wall which is measured may be a different physical property. For example, the property of the vascular wall which is measured may be a physical property of the carotid artery such a viscoelastic property. The viscoelastic property may for example be elasticity, strain amount or viscosity of the carotid artery.

The ultrasound diagnostic apparatus in the present disclosure can also effectively measure elastic modulus as the property of the vascular wall of the carotid artery, by measuring temporal variation in measured IMT values caused by vascular pulsations. Examination accuracy can be improved by measuring at the same measurement position during each examination.

### Second Embodiment

The following explains, with reference to the drawings, an ultrasound diagnostic apparatus 20 and a control method thereof relating to a second embodiment. The ultrasound diagnostic apparatus 20 is the same as the ultrasound diagnostic apparatus 10 in terms of constructing a 3D image from a plurality of transverse tomographic images acquired by scanning along the carotid artery in the longitudinal direction and in terms of measuring IMT from a longitudinal tomographic image of a measurement target region which is determined. However, the ultrasound diagnostic apparatus 20 differs from the ultrasound diagnostic apparatus 10 in terms of determining a measurement position and a measurement orientation of a measurement target region for IMT using results from both analysis of the transverse tomographic images configuring the 3D image and also analysis of a longitudinal tomographic image which is newly acquired.

<Configuration>

(General Configuration)

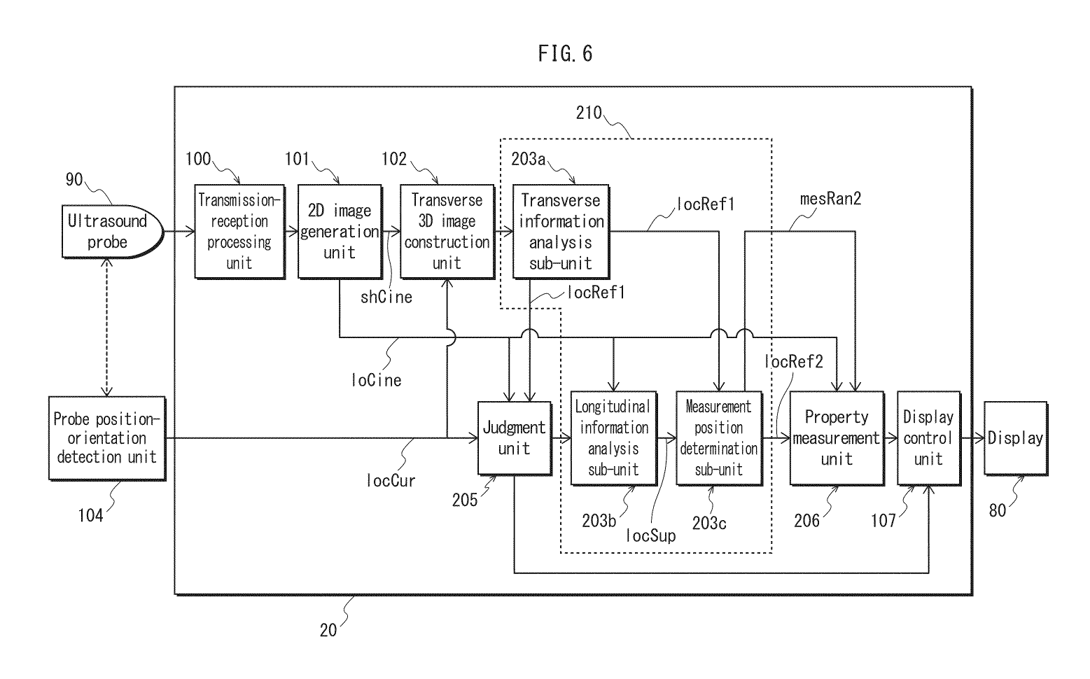

FIG. 6 is a block diagram illustrating functional configuration of the ultrasound diagnostic apparatus 20 relating to the second embodiment. As illustrated in FIG. 6, the ultrasound diagnostic apparatus 20 is configured in a manner such that an ultrasound probe 90, a probe position-orientation detection unit 104 and a display 80 are each electrically connectable to the ultrasound diagnostic apparatus 20. The ultrasound probe 90 transmits an ultrasound wave towards a subject and receives an ultrasound wave from the subject, the probe position-orientation detection unit 104 detects a position and an orientation of the ultrasound probe 90, and the display 80 displays information. FIG. 6 illustrates the ultrasound diagnostic apparatus 20 with the ultrasound probe 90, the probe position-orientation detection unit 104 and the display 80 each connected thereto.

The ultrasound diagnostic apparatus 20 includes a transmission-reception processing unit 100, a 2D image generation unit 101, a transverse 3D image construction unit 102, a display control unit 107, a transverse information analysis sub-unit 203a, a longitudinal information analysis sub-unit 203b, a measurement position determination sub-unit 203c, a judgment unit 205 and a property measurement unit 206.

The transverse information analysis sub-unit 203a, the longitudinal information analysis sub-unit 203b and measurement position determination sub-unit 203c configure a transverse-longitudinal information usage unit 210. The following focuses on explanation of configuration and operations of the judgment unit 205 and each of the sub-units configuring the transverse-longitudinal information usage unit 210.

The transmission-reception processing unit 100, the 2D image generation unit 101, the transverse 3D image construction unit 102, the display control unit 107 and the property measurement unit 206 have the same functions as elements of the same reference signs in the ultrasound diagnostic apparatus 10, and therefore explanation thereof is omitted. The ultrasound probe 90 and the probe position-orientation detection unit 104 used in the present embodiment have the same configuration as the ultrasound probe 90 and the probe position-orientation detection unit 104 used by the ultrasound diagnostic apparatus 10, and therefore explanation thereof is also omitted.

(Transverse Information Analysis Sub-Unit 203a)

The transverse information analysis sub-unit 203a analyzes a transverse 3D image in the same way as the measurement position-orientation determination unit 103 in the first embodiment and outputs first measurement position-orientation information locRef1, which includes at least information relating to a measurement target region for IMT in the carotid artery and a maximum effective plane.

(Judgment Unit 205)

The judgment unit 205 judges whether current position-orientation information locCur of a current scan plane of the ultrasound probe 90, matches the first measurement position-orientation information locRef1. When judging that the current position-orientation information locCur differs from the first measurement position-orientation information locRef1 by no greater than a threshold value, the judgment unit 205 may instruct the transmission-reception processing unit 100 and the 2D image generation unit 101 to newly acquire a longitudinal tomographic image loCine at a current position and a current orientation of the ultrasound probe 90. The transmission-reception processing unit 100 and the 2D image generation unit 101 acquire the longitudinal tomographic image loCine at the current position and the current orientation of the ultrasound probe 90, and the 2D image generation unit 101 outputs the longitudinal tomographic image loCine to the longitudinal information analysis sub-unit 203b.

(Longitudinal Information Analysis Sub-Unit 203b)

The longitudinal information analysis sub-unit 203b analyzes the longitudinal tomographic image loCine to create update information locSup for the measurement position and outputs the update information locSup to the measurement position determination sub-unit 203c. Among the measurement position and the maximum effective plane, the longitudinal information analysis sub-unit 203b updates at least the measurement position.

The following explains a method for combined use of results of analysis of transverse and longitudinal tomographic images. The transverse information analysis sub-unit 203a determines a measurement position and a measurement orientation based on a longitudinal cross-section which is generated from the transverse 3D image. Unfortunately, due to influence of vascular pulsations, detection of a measurement reference position during the above may not be sufficiently accurate. Therefore, the longitudinal information analysis sub-unit 203b detects the measurement reference position, and thus determines a measurement position, based on tunica adventitia contours extracted from the longitudinal tomographic image loCine. The tunica adventitia contours are extracted using differences between brightness values in a B-mode image, in the same way as extraction of vascular contours from each of the transverse tomographic images. In an ultrasound image which is a longitudinal tomographic image, contours appear as straight lines or gradual curves. Therefore, limitations may be set in extraction processing in order that contours are extracted which have characteristic shapes such as described above. The longitudinal information analysis sub-unit 203b determines the measurement position from the tunica adventitia contours which are extracted, using the same method as used by the transverse information analysis sub-unit 203a to determine the measurement position.

(Measurement Position Determination Sub-Unit 203c)

The measurement position determination sub-unit 203c determines second measurement position-orientation information locRef2 based on the first measurement position-orientation information locRef1 and the update information locSup. Specifically, the second measurement position-orientation information locRef2 is configured by the maximum effective plane in the first measurement position-orientation information locRef1 and the measurement position in the update information locSup.

The measurement position determination sub-unit 203c also sets measurement range information mesRan2 for IMT measurement in a scan plane determined by the second measurement position-orientation information locRef2 and outputs the measurement range information mesRan2 to the property measurement unit 206.

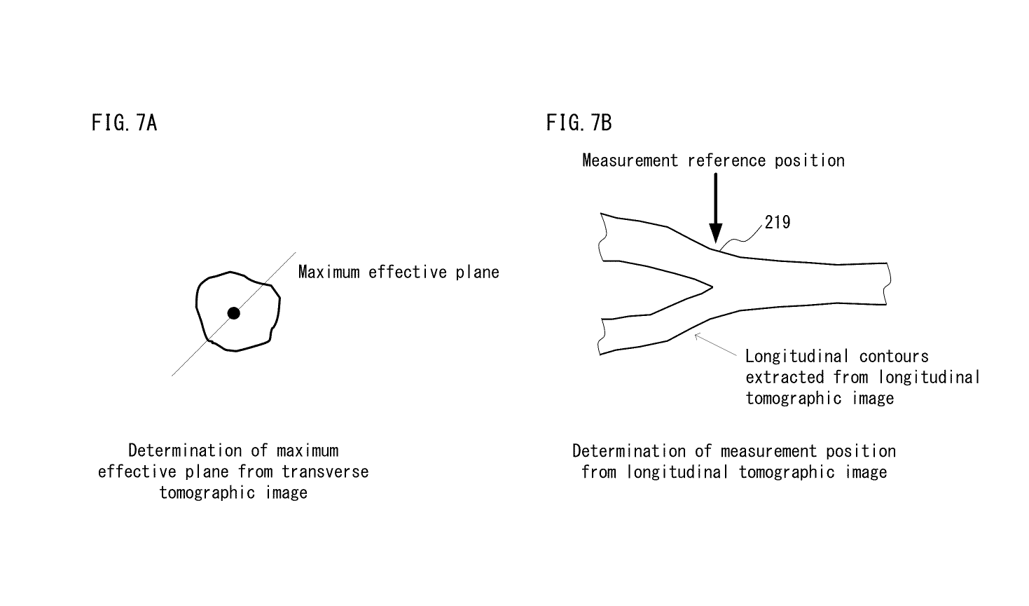

FIGS. 7A and 7B are overview diagrams provided for explaining a method for combining results of analysis of transverse and longitudinal tomographic images in the ultrasound diagnostic apparatus 20 relating to the second embodiment. In a transverse tomographic image such as illustrated in FIG. 7A, a center of contours is not easily influenced by vascular pulsations, therefore the transverse tomographic image can be used to accurately determine a maximum effective plane.

On the other hand, unevenness of longitudinal contours due to vascular pulsations does not occur in a longitudinal tomographic image such as illustrated in FIG. 7B. Therefore, the longitudinal tomographic image illustrated in FIG. 7B can be used to accurately determine a measurement reference position and thus also to accurately determine a measurement position, which is set a predetermined distance from the measurement reference position. As a consequence of the above, the maximum effective plane and the measurement position can both be accurately determined by combining results of analysis of transverse and longitudinal tomographic images.

(Property Measurement Unit 206)

The property measurement unit 206 measures IMT in a range indicated by the measurement range information mesRan2 which corresponds to the measurement target region.

<Operation>

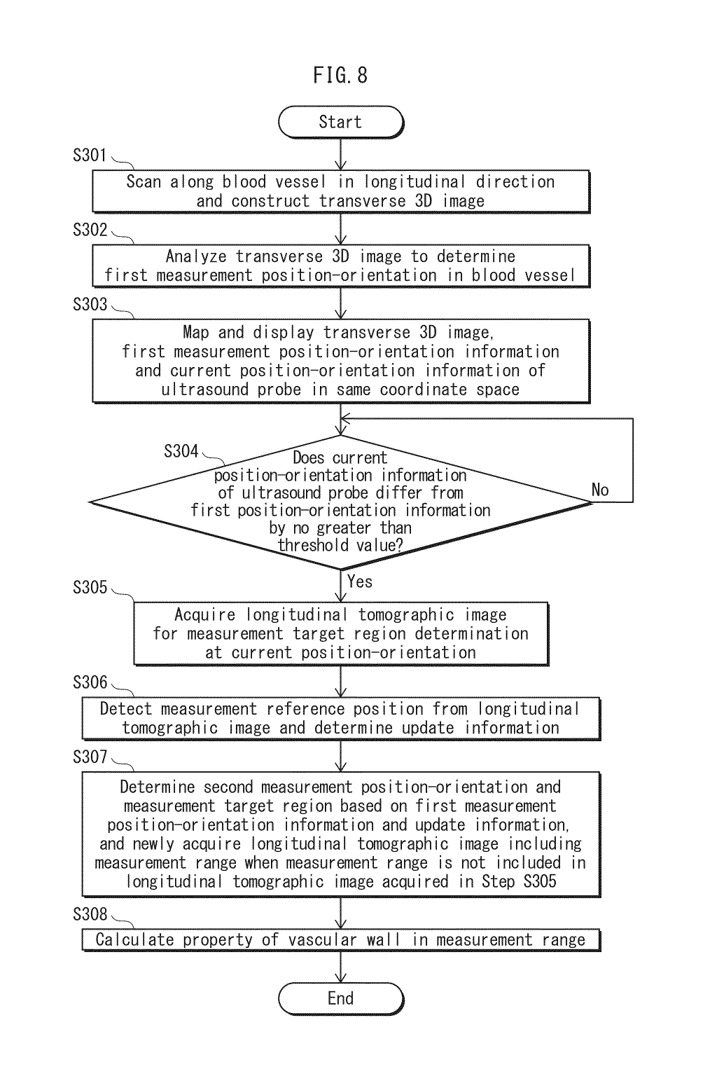

Operation of the ultrasound diagnostic apparatus 20, configured as described above, is explained below with reference to FIG. 8. FIG. 8 is a flowchart illustrating operations related to IMT measurement in the ultrasound diagnostic apparatus 20 relating to the second embodiment. Transmission of an ultrasound beam toward a carotid artery in a subject and reception of an ultrasound beam from the carotid artery is achieved using a conventional method, and therefore explanation thereof is omitted. In other words, the following explains operations up until automatic determination of measurement range for IMT and performance of IMT measurement in the measurement range.

(Step S301)

In Step S301 the ultrasound probe 90 is scanned along the blood vessel in the longitudinal direction and a transverse 3D image of vascular contours is constructed.

(Step S302)

In Step S302 the transverse 3D image is analyzed to determine first measurement position-orientation information locRef1 relating to a measurement target region.

(Step S303)

In Step S303 navigation information is displayed by mapping in a 3D space at least the transverse 3D image, the first measurement position-orientation information locRef 1 and information indicating a scan plane of the ultrasound probe 90 at a current position and a current orientation thereof. Based on the navigation information, the operator moves the ultrasound probe 90 to a first measurement position and a first measurement orientation indicated by the first measurement position-orientation information locRef 1.

(Step S304)

In Step S304 a judgment is performed to judge whether the current position and the current orientation of the ultrasound probe 90 differ from the first measurement position and the first measurement orientation respectively by no greater than a threshold value. When the judgment is affirmative processing proceeds to Step S305 and when the judgment is negative the operator continues to move the ultrasound probe 90.

(Step S305)

In Step S305 a longitudinal tomographic image loCine is acquired at the current position and the current orientation of the ultrasound probe 90.

(Step S306)

In Step S306 vascular contours of the tunica adventitia are extracted from the longitudinal tomographic image loCine acquired in Step S305, a measurement reference position is detected and update information locSup is determined. Using the same method as performed by the transverse information analysis sub-unit 203a, a measurement position is determined from the vascular contours which are extracted. In other words, a measurement position is redetermined by analyzing the longitudinal tomographic image using the same method as the measurement position-orientation determination unit 103 in the first embodiment.

(Step S307)

In Step S307 second measurement position-orientation information locRef2 is determined based on the first measurement position-orientation information locRef1 and the update information locSup. Measurement range information mesRan2 is also determined, indicating a measurement range for IMT.

The processing flow described above is for a configuration in which, once a longitudinal tomographic image for measurement is determined in Step S305, update information locSup is determined in Step S306 based on the longitudinal tomographic image. However, when a position and an orientation indicated by the first measurement position-orientation information locRef1 and differ respectively from a position and an orientation indicated by the second measurement position-orientation information locRef2 by a large amount, a case may arise in which the longitudinal tomographic image acquired in Step S305 does not include the measurement range corresponding to the second measurement position-orientation information locRef2.

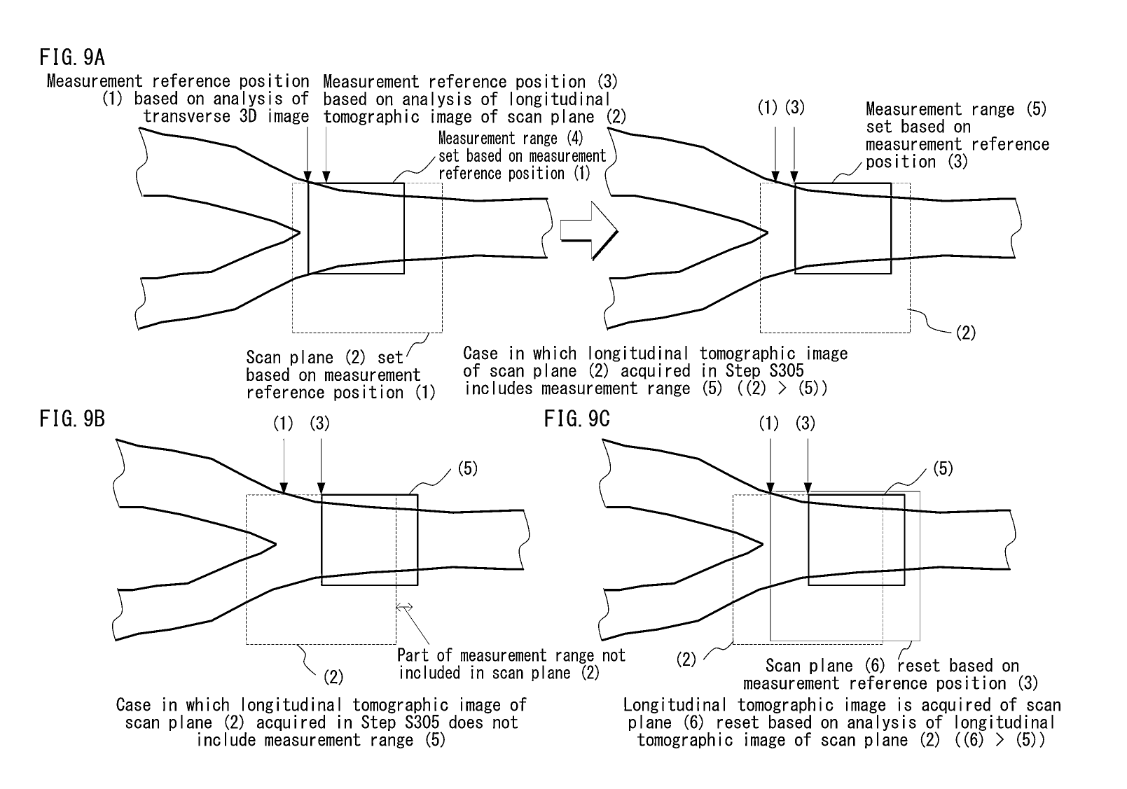

FIGS. 9A-9C are provided for explaining relationship between a longitudinal tomographic image and a measurement target region in the ultrasound diagnostic apparatus 20 relating to the second embodiment.

FIG. 9A illustrates a case in which the measurement range is included in the longitudinal tomographic image acquired in Step S305. In the left side of FIG. 9A, reference sign (1) indicates a measurement reference position which is calculated based on analysis of the transverse 3D image. Reference sign (2) indicates a scan plane which is set based on the measurement reference position (1). Reference sign (3) indicates a measurement reference position which is calculated based on analysis of the longitudinal tomographic image. Reference sign (4) indicates a measurement range which is set based on the measurement reference position (1).

In the right side of FIG. 9A, reference sign (5) indicates a measurement range which is set based on the measurement reference position (3). IMT is measured in the measurement range (5). In the above case, the scan plane (2) includes the measurement range (5), and therefore a problem does not occur during IMT measurement.

On the other hand, in a case illustrated in FIG. 9B a difference between the measurement reference position (1) and the measurement reference position (3) is large. Consequently, in the above case the scan plane (2) does not include the measurement range (5), and therefore IMT cannot be measured correctly.

As illustrated in FIG. 9C, in order to prevent the above problem from occurring, once the measurement range (5) has been determined based on the longitudinal tomographic image acquired in Step S305, a scan plane (6) may be set based on the measurement reference position (3) in order that the scan plane (6) includes the measurement range (5). A longitudinal tomographic image of the scan plane (6) may be newly acquired and IMT may be measured in Step S308 based on the longitudinal tomographic image which is newly acquired.

During the above, position of the scan plane which is reset is presented on the navigation screen. For example, by indicating position of the scan plane which is initially set using flashing display and indicating position of the scan plane which is reset using non-flashing display, differentiation between the two different scan planes is possible. Alternatively, in order that navigation can be performed as a series of operations up until resetting of the scan plane, once the current position and the current orientation differ from the first measurement position and the first measurement orientation respectively by no greater than a threshold value, a longitudinal tomographic image may be acquired at the current position, resetting of the scan plane may be performed, the navigation screen may be updated to reflect the reset scan plane and the operator may be guided to the reset scan plane. IMT measurement is performed based on a longitudinal tomographic image acquired at the reset scan plane.

(Step S308)

In Step S308, based on the longitudinal tomographic image loCine, IMT is measured in the measurement range indicated by the measurement range information mesRan2, which corresponds to the measurement target region.

<Specific Examples of Diagnostic Methods Measuring IMT>

The following explains specific examples of diagnostic methods in which IMT is measured. There are broadly speaking two different types of diagnostic method with regards to IMT in the carotid artery. In one method which is for screening purposes such as in a health check-up, a degree of arteriosclerosis is judged by measuring IMT at a predetermined position in the carotid artery. The above measurement is referred to as an IMT measurement. In another method which is for precise diagnostic purposes, a search is performed for a position at which IMT has a maximum value in the CCA and the bulb, or alternatively in the ICA or the ECA. IMT is subsequently measured only at the position of the maximum value and positions within a predetermined distance thereof, for example positions within 1 cm either side of the position of the maximum value. The above measurement is referred to as a Max-IMT measurement.

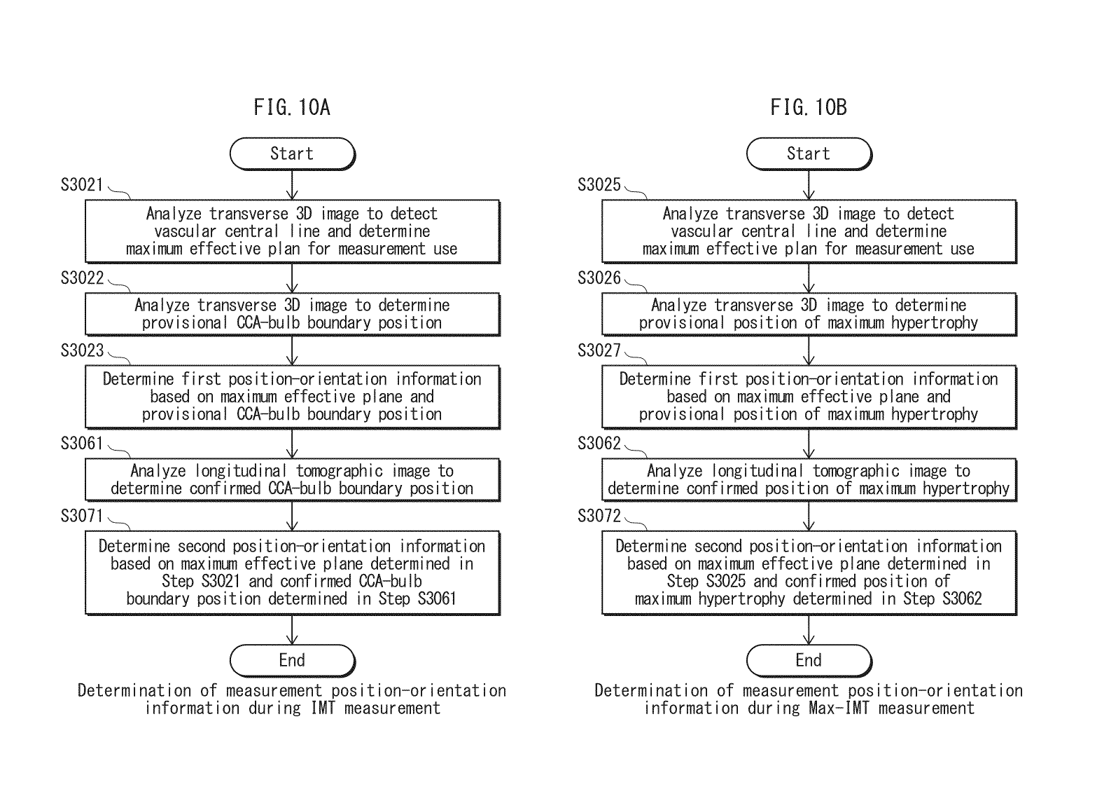

FIG. 10A is a flowchart illustrating determination of measurement position-orientation information during IMT measurement and FIG. 10B is a flowchart illustrating determination of measurement position-orientation during Max-IMT measurement.

(Determination of Measurement Position-Orientation Information During IMT Measurement)

First, in Step S3021 of IMT measurement, a transverse 3D image is analyzed to detect a vascular central line in order to determine a maximum effective plane.

Next, in Step S3022 the transverse 3D image is analyzed to determine a provisional position of the CCA-bulb boundary 219.

In Step S3023 first measurement position-orientation information is determined based on the maximum effective plane and the provisional position of the CCA-bulb boundary 219.

In Step S3061 a longitudinal tomographic image, which is acquired by the operator performing operation in accordance with navigation, is analyzed to determine a confirmed position of the CCA-bulb boundary 219.

Finally, in Step S3071 second measurement position-orientation information is determined based on the maximum effective plane determined in Step S3021 and the confirmed position of the CCA-bulb boundary 219 determined in Step S3061.

(Determination of Measurement Position-Orientation Information During Max-IMT Measurement)

First, in Step S3025 of Max-IMT measurement, a transverse 3D image is analyzed to detect a vascular central line and to determine a maximum effective plane for use in measurement.

Next, in Step S3026 the transverse 3D image is analyzed to determine a provisional position of maximum hypertrophy of the intima-media complex. A position of maximum hypertrophy may be detected in each part of the carotid artery such as the CCA and the bulb, or alternatively in the ICA or the ECA. Further alternatively, the carotid artery may be divided into a plurality of sections along the scan direction thereof, and a position of maximum hypertrophy may be detected in each of the sections. In a configuration such as described above, a position of maximum hypertrophy is detected in each of the parts or sections, thus a plurality of positions of maximum hypertrophy are set. In an alternative configuration, instead of detecting one or more points of maximum hypertrophy, each part in which IMT exceeds a predetermined threshold value may be detected.

Next, in Step S3027 first measurement position-orientation information is determined, based on the maximum effective plane and the provisional position of maximum hypertrophy, in order that a scan plane is the maximum effective plane at the point of maximum hypertrophy.

In Step S3062 a longitudinal tomographic image, which is acquired by the operator performing operation in accordance with navigation, is analyzed to determine a confirmed position of maximum hypertrophy.

Finally, in Step S3072 second measurement position-orientation information is determined based on the maximum effective plane determined in Step S3025 and the confirmed position of maximum hypertrophy determined in Step S3062.

In order to perform repeated examinations over time or confirm effectiveness of medication, preferably measurement during each subsequent examination should be performed at the same position as that of a point of maximum hypertrophy measured during an initial examination. The Max-IMT measurement method described above is highly effective for accurately determining position of the point of maximum hypertrophy.

Alternatively, a plurality of maximum effective planes may be set for performing measurement and measurement position-orientation information may be presented to the operator in a manner such as to guide the operator to measure the plurality of maximum effective planes in order. Particularly in Max-IMT measurement, which targets a site where a plaque is formed, by performing measurements for a plurality of maximum effective planes, a 3D shape of the plaque can be more accurately determined. For example, a maximum effective plane found to have a maximum IMT through analysis of a transverse 3D image may be used as a reference, and maximum effective planes differing by a predetermined angle may also be targets for measurement.

<Effects>

Vascular pulsations cause unevenness of contours in a longitudinal cross-section image which is generated from a transverse 3D image. Consequently, accuracy is reduced when determining a measurement reference position by analyzing the contours in the longitudinal cross-section image, and therefore a measurement position cannot be accurately determined. When determining the measurement reference position, reduction in accuracy such as described above presents a problem.

In response to the above problem, the ultrasound diagnostic apparatus 20 relating to the second embodiment determines a position and an orientation at which a longitudinal tomographic image can be acquired including a measurement target region for IMT, based on a transverse 3D image, and subsequently determines the measurement target region based on the longitudinal tomographic image which is newly acquired. In a conventional configuration in which a measurement target region for IMT is determined based on a longitudinal cross-section generated from a transverse 3D image, a problem occurs of a measurement reference position, which is used as a reference for determining the measurement target region for IMT in terms of the longitudinal direction, varying due to vascular pulsations. However, the above problem does not occur in the ultrasound diagnostic apparatus 20 due to the configuration described above. Therefore, through the ultrasound diagnostic apparatus 20 the operator can acquire a longitudinal tomographic image including the measurement target region and thus can accurately measure IMT.

Furthermore, through determination of measurement position using both transverse and longitudinal tomographic images, the ultrasound diagnostic apparatus 20 provides improved IMT measurement accuracy. Consequently, accuracy and reproducibility can be significantly improved for IMT measurement performed by ultrasound diagnosis of a blood vessel.

### Modified Examples

The above is an explanation of the ultrasound diagnostic apparatus 20 relating to the present embodiment. The ultrasound diagnostic apparatus in the present disclosure is of course not limited to the ultrasound diagnostic apparatus explained in the present embodiment and various modified examples of the ultrasound diagnostic apparatus in the present embodiment are also possible, such as explained below.

(1) The operations for Max-IMT measurement in FIG. 10B are explained using an example in which second measurement-orientation information is determined using both transverse and longitudinal tomographic images, however the operations in FIG. 10B may also be adapted for use in the ultrasound diagnostic apparatus 10. In the ultrasound diagnostic apparatus 10, measurement position-orientation information is determined based only on analysis of transverse tomographic images, therefore only operation steps for determining first measurement position-orientation information are necessary.

(2) The ultrasound diagnostic apparatus 20 may alternatively have a configuration in which the operator can switch between IMT measurement illustrated in FIG. 10A and Max-IMT measurement illustrated in FIG. 10B. When switching between IMT measurement and Max-IMT measurement, a switching signal is input into the transverse-longitudinal information usage unit 210 and the transverse-longitudinal information usage unit 210 switches between modes for IMT measurement and Max-IMT measurement based on the signal which is input.

(3) An objective in the present embodiment is navigation. However, the method described in the present embodiment for accurately determining a position using both transverse and longitudinal tomographic images may be used for a different objective. For example, a longitudinal tomographic image may be used to accurately measure position, relative to a measurement reference position, of a plaque which is detected in a transverse 3D image.

### Third Embodiment

The following explains, with reference to the drawings, an ultrasound diagnostic apparatus 30 and a control method thereof relating to a third embodiment. The ultrasound diagnostic apparatus 30 is the same as the ultrasound diagnostic apparatus 10 in terms of constructing a 3D image from a plurality of transverse tomographic images, which are acquired by scanning along the carotid artery in the longitudinal direction, and in terms of measuring IMT from a longitudinal tomographic image of a measurement target region which is determined. However, the ultrasound diagnostic apparatus 30 differs from the ultrasound diagnostic apparatus 10 in terms of analyzing a property of a vascular wall, such as plaque surface area, plaque volume, percentage area stenosis or percentage diameter stenosis, from the 3D image based on the transverse tomographic images configuring the 3D image.

<Configuration>

(General Configuration)

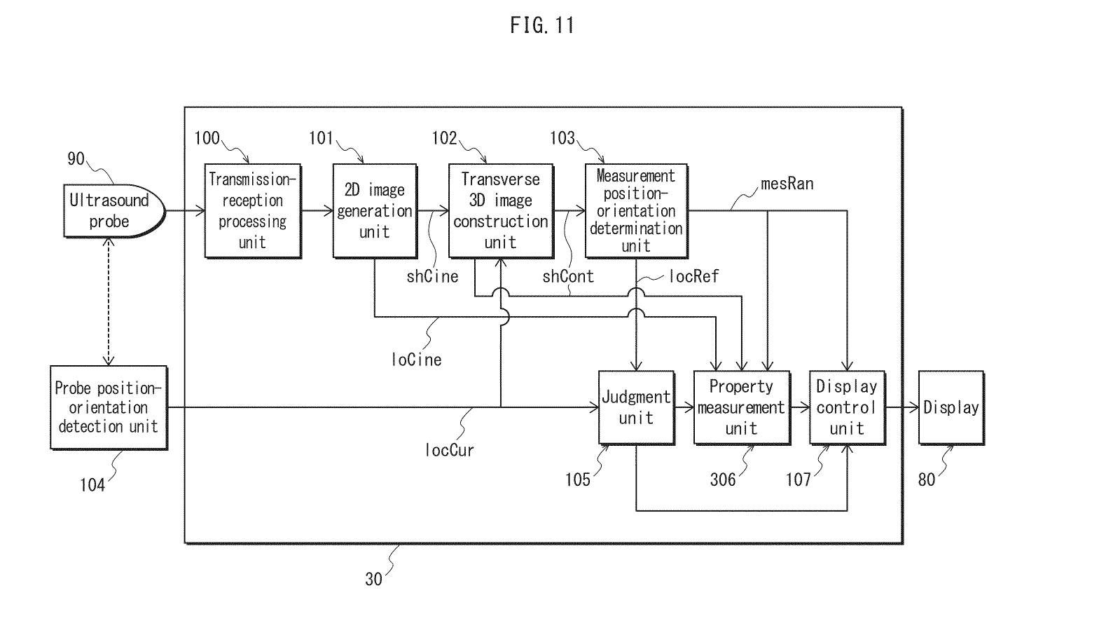

FIG. 11 is a block diagram illustrating functional configuration of the ultrasound diagnostic apparatus 30 relating to the third embodiment. As illustrated in FIG. 11, the ultrasound diagnostic apparatus 30 is configured in a manner such that an ultrasound probe 90, a probe position-orientation detection unit 104 and a display 80 are each electrically connectable to the ultrasound diagnostic device 30. The ultrasound probe 90 transmits an ultrasound wave towards a subject and receives an ultrasound wave from the subject, the probe position-orientation detection unit 104 detects a position and an orientation of the ultrasound probe 90, and the display 80 displays information. FIG. 11 illustrates the ultrasound diagnostic apparatus 30 with the ultrasound probe 90, the probe position-orientation detection unit 104 and the display 80 each connected thereto.

The ultrasound diagnostic apparatus 30 includes a transmission-reception processing unit 100, a 2D image generation unit 101, a transverse 3D image construction unit 102, a measurement position-orientation determination unit 103, a judgment unit 105, a display control unit 107 and a property measurement unit 306. The following explanation focuses on operations of the measurement position-orientation determination unit 103 and the property measurement unit 306. The transmission-reception processing unit 100, the 2D image generation unit 101, the transverse 3D image construction unit 102, the judgment unit 105 and the display control unit 107 in the ultrasound diagnostic apparatus 30 have the same functions as elements of the same reference signs in the ultrasound diagnostic apparatus 10, and therefore explanation thereof is omitted. The ultrasound probe 90 and the probe position-orientation detection unit 104 used by the ultrasound diagnostic apparatus 30 have the same configuration as the ultrasound probe 90 and the probe position-orientation detection unit 104 used by the ultrasound diagnostic apparatus 10, and therefore explanation thereof is also omitted.

(Measurement Position-Orientation Determination Unit 103)

The measurement position-orientation determination unit 103 analyzes shape of lumen-intima interface contours and media-adventitia interface contours in a transverse 3D image, detects a position of maximum hypertrophy for IMT in a longitudinal cross-section of a blood vessel and identifies the position as a plaque at which hypertrophy of the intima-media occurs.

(Property Measurement Unit 306)

The property measurement unit 306 acquires a 2D image loCine from the 2D image generation unit 101 and measures IMT in a range indicated by the measurement range information mesRan in the same way as in the ultrasound diagnostic apparatus 10. IMT measurement from the 2D image loCine is the same as in the ultrasound diagnostic apparatus 10, therefore explanation thereof is omitted. The property measurement unit 306 also acquires the 3D image shCont of vascular contours from the transverse 3D image construction unit 102, analyzes a property of the vascular wall from the 3D image shCont, and outputs measurement results to the display control unit 107. Plaque surface area, plaque volume, percentage area stenosis or percentage diameter stenosis can for example be measured from the 3D image shCont as the property of the vascular wall.

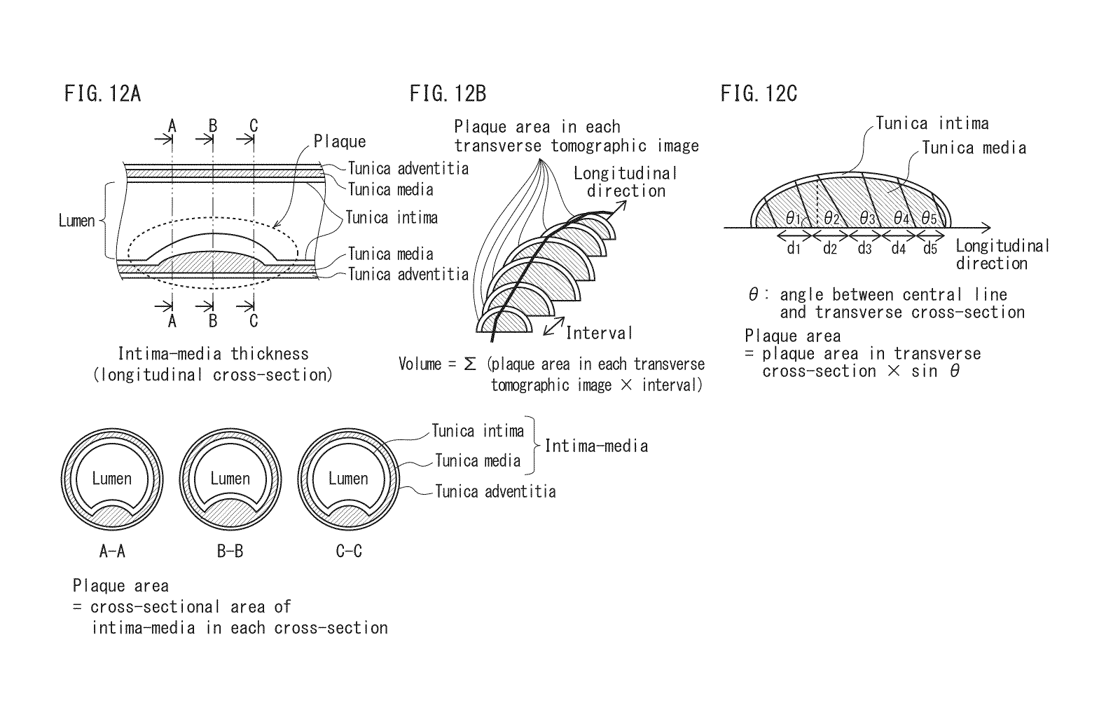

FIGS. 12A-12C are provided for explaining a method for measuring plaque volume of the ultrasound diagnostic apparatus 30 relating to the third embodiment. As explained above, a localized area in which hypertrophy of the intima-media causes IMT to exceed a predetermined value is referred to as a plaque. FIG. 12A illustrates change in structure of the vascular wall in the above situation.

In order to measure a plaque, the measurement position-orientation determination unit 103 determines a position of maximum hypertrophy of the intima-media in the carotid artery, as illustrated in FIG. 12A. Next, the property measurement unit 306 calculates intima-media cross-sectional area in each of a plurality of transverse tomographic images close to the position of maximum hypertrophy. FIG. 12A illustrates an example in which intima-media cross-sectional area is calculated in transverse tomographic images which are cross-sections at three different locations indicated by lines A-A, B-B and C-C. In order to calculate cross-sectional area of the intima-media in each of the cross-sections, the property measurement unit 306 first uses an image processing method, such as edge detection processing or an active contour method, to extract a lumen-intima interface contour and a media-adventitia interface contour based on the transverse tomographic image corresponding to the cross-section. Next, the property measurement unit 306 calculates an area included between the lumen-intima interface contour and the media-adventitia interface contour, and thus calculates the cross-sectional area of the intima-media in the cross-section as a plaque area.

Next, plaque volume can be calculated as illustrated in FIG. 12B by multiplying plaque area in each of the cross-sections by a distance to a neighboring cross-section in order to perform integration of plaque area in the longitudinal direction.

In a situation in which each of the transverse cross-sections is inclined by an angle θ relative to the longitudinal direction, plaque volume can be calculated accurately by integrating in the longitudinal direction after performing a correction by multiplying the plaque area in each of the cross-sections by sin θ.

<Operation>

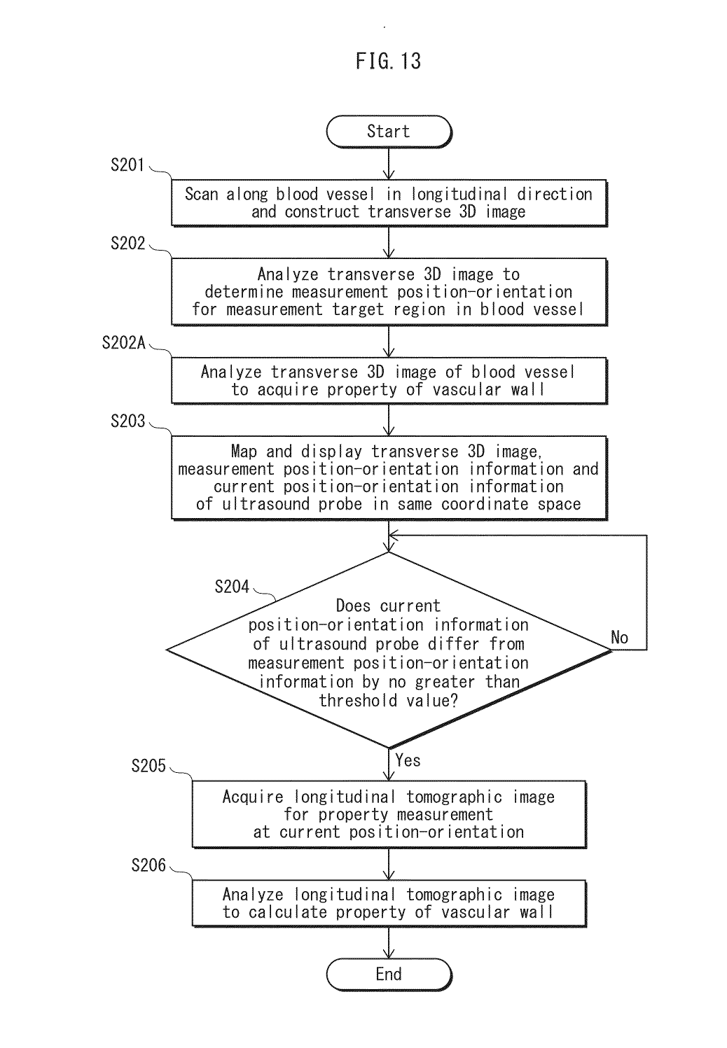

Operation of the ultrasound diagnostic apparatus 30, configured as described above, is explained below with reference to FIG. 13. FIG. 13 illustrates a flowchart for the ultrasound diagnostic apparatus 30 relating to the third embodiment. Operations in Steps S201, S202 and from Step S203 onward are the same as in the ultrasound diagnostic apparatus 10, therefore explanation thereof is omitted.

(Step S202A)

In Step S202A the measurement position-orientation determination unit 103 analyzes shape of lumen-intima interface contours and media-adventitia interface contours in a transverse 3D image, detects a position of maximum hypertrophy for IMT in a longitudinal cross-section of a blood vessel and identifies the position of maximum hypertrophy as a plaque at which hypertrophy of the intima-media occurs.

Next, the property measurement unit 306 calculates plaque area in each of a plurality of transverse cross-sections close to the position of maximum hypertrophy for IMT in the longitudinal cross-section. The property measurement unit 306 calculates the plaque are in each of the cross-sections using shape of lumen-intima interface contours and media-adventitia interface contours in the transverse 3D image. As described above, the plaque area in each of the cross-sections is calculated by calculating a cross-sectional area of the intima-media between a lumen-intima interface contour and a media-adventitia interface contour in the cross-section. Next, plaque volume is calculated by multiplying the plaque area in each of the cross-sections by a distance to a neighboring cross-section in order to perform integration of plaque area in the longitudinal direction.

The plaque volume which is calculated is presented to the operator, through display by the display 80, as navigation information mapped to the same coordinate space as the transverse 3D image, the position of maximum hypertrophy, and the lumen-intima interface contours and the media-adventitia interface contours in each of the transverse cross-sections. 3D contours of the plaque can be acquired by interpolating contours in a plurality of longitudinal images which are acquired. Alternatively, volume may for example be based on results of the interpolation.

<Effects>

The ultrasound diagnostic apparatus 30 relating to the third embodiment detects a position of maximum hypertrophy by analyzing a transverse 3D image, and calculates plaque volume by first calculating plaque area in each of a plurality of transverse cross-sections close to the position of maximum hypertrophy and by subsequently multiplying the plaque area in each of the cross-sections by a distance to a neighboring cross-section in order to integrate plaque area in the longitudinal direction. In other words, the ultrasound diagnostic apparatus 30 can accurately measure size of a plaque. Quantitative evaluation during plaque diagnosis is difficult, but through the above configuration objectiveness of the quantitative evaluation can be improved. Therefore, treatment such as medicinal treatment or surgical treatment to remove the plaque can be performed appropriately based on the plaque diagnosis. Measurement of plaque volume is particularly effective in determining, at an early stage, effectiveness of medicinal treatment in causing reduction in size of a plaque.

### Modified Examples

The above is an explanation of the ultrasound diagnostic apparatus 30 relating to the present embodiment. The ultrasound diagnostic apparatus in the present disclosure is of course not limited to the ultrasound diagnostic apparatus explained in the present embodiment and various modified examples of the ultrasound diagnostic apparatus in the present embodiment are also possible, such as explained below.

(1) The ultrasound diagnostic apparatus 30 is explained using plaque area and plaque volume as examples of properties of the vascular wall which are acquired from the 3D image and analyzed. However, other properties of the vascular wall, which can be acquired from the 3D image, may also be calculated, such as percentage area stenosis or percentage diameter stenosis. For example, percentage area stenosis can be calculated by calculating a ratio of a cross-sectional area within the lumen-intima interface when a plaque is present with respect to a cross-sectional area within the lumen-intima interface estimated for when the plaque is not present. The cross-sectional area within the lumen-intima interface when the plaque is not present can be estimated by extrapolation of the lumen-intima interface in parts of a transverse tomographic image in which the plaque is not present.

(2) The ultrasound diagnostic apparatus 30 analyzes plaque area and plaque volume from a 3D image, which is constructed from a plurality of transverse tomographic images acquired by performing a hand scan in the longitudinal direction. However, in an alternative configuration plaque volume may be measured using a plurality of longitudinal tomographic images acquired at equal intervals over an entire extent of a plaque. In the above configuration, longitudinal tomographic images required for measuring plaque volume may be determined and navigation may be performed in a manner such that the longitudinal tomographic images are acquired.

(3) In order that temporal change of a plaque can be observed, navigation may alternatively be performed based on positional relationship of a 3D image and a scan plane in an examination which is used as a reference.

For example, an initial examination may be used as a reference by at least storing a positional relationship of a 3D image and a scan plane in the initial examination. More specifically, the positional relationship which is stored may be a distance from a measurement reference position in the carotid artery, such as the Bif 217 or the CCA-bulb boundary 219, and an orientation of the scan plane relative to a central line of the CCA 213, or an orientation of the scan plane relative to a flat plane passing through points on a central line of the CCA 213 and the ICA 215, or the CCA 213 and the ECA 216. During a subsequent examination, once a scan is performed in the longitudinal direction and a 3D image is constructed, a reference scan plane which is stored is displayed superimposed on the 3D image. Through the above, the operator can easily acquire an ultrasound image at the reference scan plane.

Consequently, when measuring presence or thickness of carotid artery plaques, which are a major cause of cerebral infarction, a measurement position for a plaque can be determined in a transverse direction cross-section of the carotid artery, and the plaque can be measured at the same position during each examination, allowing observation of temporal change in size of the plaque.

### Fourth Embodiment

The following explains, with reference to the drawings, an ultrasound diagnostic apparatus 40 and a control method thereof relating to a fourth embodiment. The ultrasound diagnostic apparatus 40 is the same as the ultrasound diagnostic apparatus 10 in terms of constructing a 3D image from a plurality of transverse tomographic images, which are acquired by scanning along the carotid artery in the longitudinal direction, and in terms of measuring IMT from a longitudinal tomographic image of a measurement target region which is determined. However, the ultrasound diagnostic apparatus 40 differs from the ultrasound diagnostic apparatus 10 in terms that the ultrasound diagnostic apparatus 40 is configured such that an ultrasound probe 91, which is provided with a plurality of transducer columns thereon, is connectable to the ultrasound diagnostic apparatus 40. Each of the transducer columns has a plurality of ultrasound transducers arranged in a column direction and the transducer columns are arranged in a row direction which is perpendicular to the column direction. The ultrasound diagnostic apparatus 40 also differs in terms that provision of a position-orientation detection unit 104 for detecting a position and an orientation of the ultrasound probe is not necessary.

<Configuration>

(General Configuration)

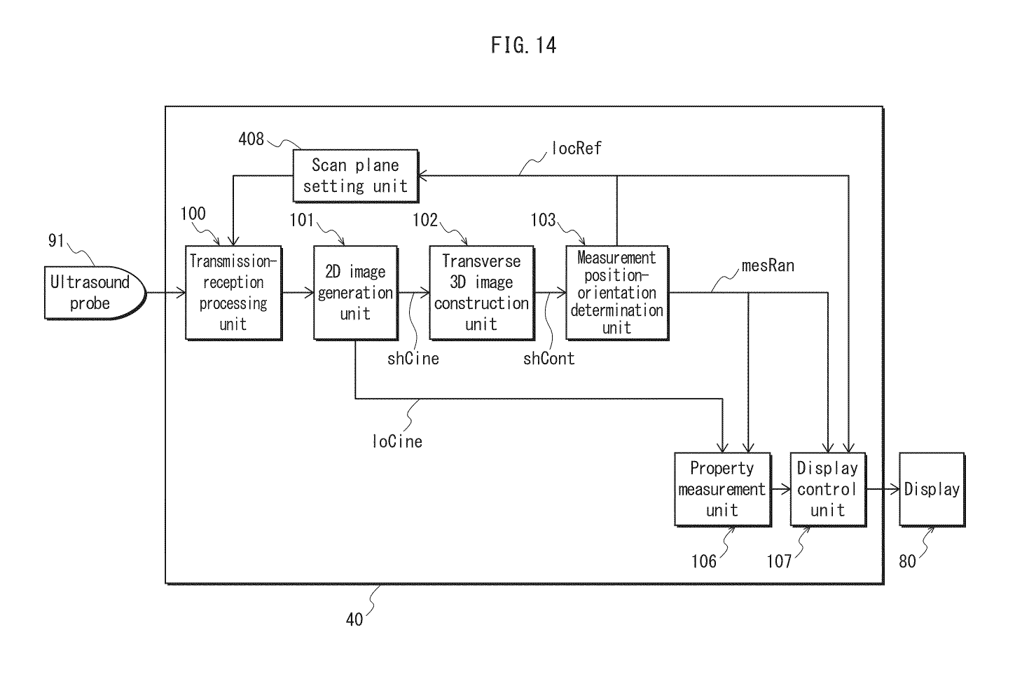

FIG. 14 is a block diagram illustrating functional configuration of the ultrasound diagnostic apparatus 40 relating to the fourth embodiment. As illustrated in FIG. 14, the ultrasound diagnostic apparatus 40 is configured in a manner such that the ultrasound probe 91 and a display 80 are each electrically connectable to the ultrasound diagnostic device 40. The ultrasound probe 91 transmits an ultrasound wave toward a subject and receives an ultrasound wave from the subject, and the display 80 displays information. FIG. 14 illustrates the ultrasound diagnostic apparatus 40 with the ultrasound probe 91 and the display 80 each connected thereto.

The ultrasound diagnostic apparatus 40 includes a transmission-reception processing unit 100, a 2D image generation unit 101, a transverse 3D image construction unit 102, a measurement position-orientation determination unit 103, a property measurement unit 106, a display control unit 107 and a scan plane setting unit 408. The following focuses on explanation of configuration and operation of the scan plane setting unit 408. The ultrasound probe 91 is also explained. The transmission-reception processing unit 100, the 2D image generation unit 101, the transverse 3D image construction unit 102, the measurement position-orientation determination unit 103, the property measurement unit 106 and the display control unit 107 in the ultrasound diagnostic apparatus 40 have the same functions as elements of the same reference signs in the ultrasound diagnostic apparatus 10, and therefore explanation thereof is omitted.

(Ultrasound Probe 91)

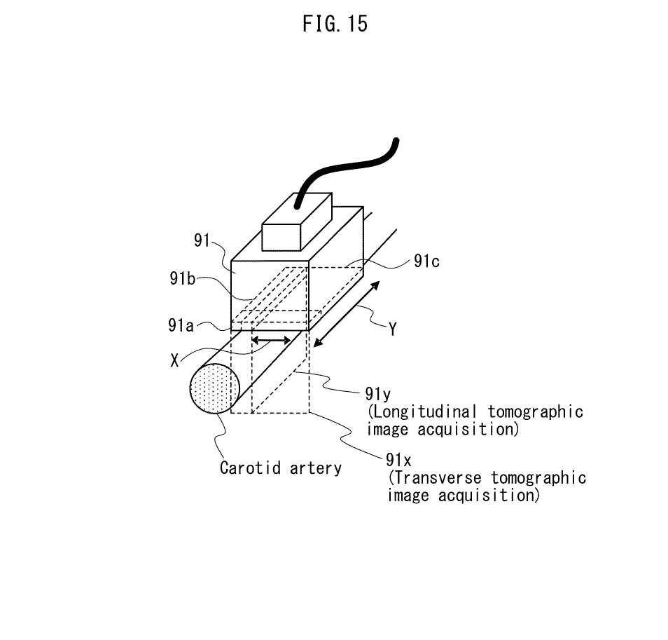

FIG. 15 is a schematic diagram illustrating the ultrasound probe 91 used by the ultrasound diagnostic apparatus 40 relating to the fourth embodiment. As illustrated in FIG. 15, a plurality of transducer columns 91a, which each have a plurality of piezoelectric elements arranged in straight line in a column direction X, are provided on the ultrasound probe 91. The transducer columns 91a are arranged in a row direction Y which is perpendicular to the column direction X, thus forming a matrix of transducers 91c in a 2D array.

The ultrasound probe 91 receives a transmission signal supplied from the transmission-reception processing unit 100 as a pulse or continuous electrical signal, and converts the transmission signal into a pulse or continuous ultrasound wave. While the transducers 91c are in contact with skin surface of a subject, the ultrasound probe 91 emits an ultrasound beam from the skin surface toward the carotid artery. In order to acquire a 2D image of a transverse cross-section of the carotid artery, the ultrasound probe 91 is for example positioned so that the column direction X of the transducers 91c arranged in the 2D array is perpendicular to the longitudinal direction of the carotid artery. In order to form a scan plane 91x perpendicular to the longitudinal direction, at least one of the transducer columns 91a is driven to emit an ultrasound beam. In the above situation, if a plurality of the transducer columns 91a are driven a scan plane is formed by beamforming. The ultrasound probe 91 also receives an ultrasound echo signal which is an ultrasound wave reflected from the subject. The ultrasound probe 91 converts the ultrasound echo signal to an electrical signal through the transducer columns 91a and supplies the electrical signal to the transmission-reception processing unit 100.

In order to acquire a plurality of transverse tomographic images of the carotid artery, the transducer columns 91a which are driven, are electrically scanned in the row direction Y, and thus are scanned along the carotid artery in the longitudinal direction. With the transducers 91c of the ultrasound probe 91 in contact with the skin surface, the transducer columns 91a which are driven, transmit an ultrasound beam scanning in the row direction Y, which is the longitudinal direction of the carotid artery.

(Transmission-Reception Processing Unit 100) A plurality of scan planes 91x are formed perpendicular to the longitudinal direction, and by driving the transducer columns 91a to scan a plurality of times along the row direction Y (referred to below as a row direction scan), reception signals are successively generated for carotid artery cross-sections in a plurality of frames corresponding one-to-one to the scan planes 91x. The transmission-reception processing unit 100 supplies the reception signal in each of the plurality of frames to the 2D image generation unit 101.

(2D Image Generation Unit 101)

For each of the frames, the 2D image generation unit 101 generates a 2D image shCine, which is a transverse tomographic image of the carotid artery corresponding to the frame, based on the reception signal in the frame. The 2D image generation unit 101 supplies the 2D images shCine to the 3D image construction unit 102. Each of the 2D images shCine is supplied to the transverse 3D image construction unit 102 with a column number, indicating a column for which the 2D image shCine is acquired in the row direction scan.

(Transverse 3D Image Construction Unit 102)

The transverse 3D image construction unit 102 extracts vascular contours of the carotid artery from each of the 2D images shCine. The transverse 3D image construction unit 102 subsequently constructs a 3D image of the carotid artery by mapping the vascular contours extracted from each of the transverse tomographic images in a 3D space, based on position of a scan plane of which the transverse tomographic image is acquired. During the above, the transverse 3D image construction unit 102 constructs the 3D image of the carotid artery by connecting the vascular contours in an order corresponding to order in which the transverse tomographic images, from which the vascular contours are extracted, are acquired during the row direction scan. The transverse 3D image construction unit 102 outputs the vascular contours mapped in the 3D space and coordinates shCont of the vascular contours to the measurement position-orientation determination unit 103.

(Measurement Position-Orientation Determination Unit 103)

The measurement position-orientation determination unit 103 analyzes a transverse 3D image in the same way as the measurement position-orientation determination unit 103 in the first embodiment and outputs measurement position-orientation information locRef including at least information relating to a measurement target region for IMT in the carotid artery and a maximum effective plane.

(Scan Plane Setting Unit 408)

The scan plane setting unit 408 determines a scan plane for the ultrasound probe 91 in order to acquire a longitudinal tomographic image including the measurement target region for IMT, based on the measurement position-orientation information locRef. The scan plane setting unit 408 instructs the transmission-reception processing unit 100 to perform transmission processing and reception processing in order to acquire the longitudinal tomographic image for property measurement at the scan plane which is determined.

Specifically, the ultrasound probe 91 is positioned at the same position as when acquiring the transverse tomographic images, and the ultrasound probe 91 is orientated such that the column direction X of the transducers 91c, arranged as a matrix in a 2D array, is perpendicular to the longitudinal direction of the carotid artery. Next, in order to form a scan plane 91y, which is parallel to the longitudinal direction, at least one transducer row 91b, which is parallel to the row direction Y and the longitudinal direction, is driven to emit an ultrasound beam. In the above situation, if a plurality of transducer rows 91b are driven the scan plane 91y is formed by beamforming. The ultrasound probe 91 also receives an ultrasound sound echo signal which is an ultrasound wave reflected from the subject, converts the ultrasound echo signal to an electrical signal through the transducer rows 91b, and supplies the electrical signal to the transmission-reception processing unit 100.

Upon receiving the reception signal from the transmission-reception processing unit 100, the 2D image generation unit 101 generates a 2D image loCine, which is a longitudinal tomographic image, based on the reception signal and supplies the 2D image loCine to the property measurement unit 106, which is explained below.

(Property Measurement Unit 106)

The property measurement unit 106 acquires the 2D image loCine from the 2D image generation unit 101 and measures IMT in a range indicated by the measurement range information mesRan.

Through the above, the property measurement unit 106 measures IMT based on the longitudinal tomographic image loCine acquired at the scan plane 91y, which is determined by the scan plane setting unit 408 based on the measurement position-orientation information locRef.

(Display Control Unit 107)

The display control unit 107 receives the measurement position-orientation information locRef, which indicates the scan plane, and the measurement range information mesRan, which indicates the measurement range for IMT, from the measurement position-orientation determination unit 103. The display control unit 107 controls the display 80 to display the scan plane and the measurement range for IMT superimposed on the transverse 3D image.

The display control unit 107 also receives information indicating results of IMT measurement from the property measurement unit 106 and controls the display 80 to display the results. During the above, a more user friendly configuration is achieved by displaying the IMT measurement range 212, in which IMT measurement is performed, with the 3D image.

<Operation>

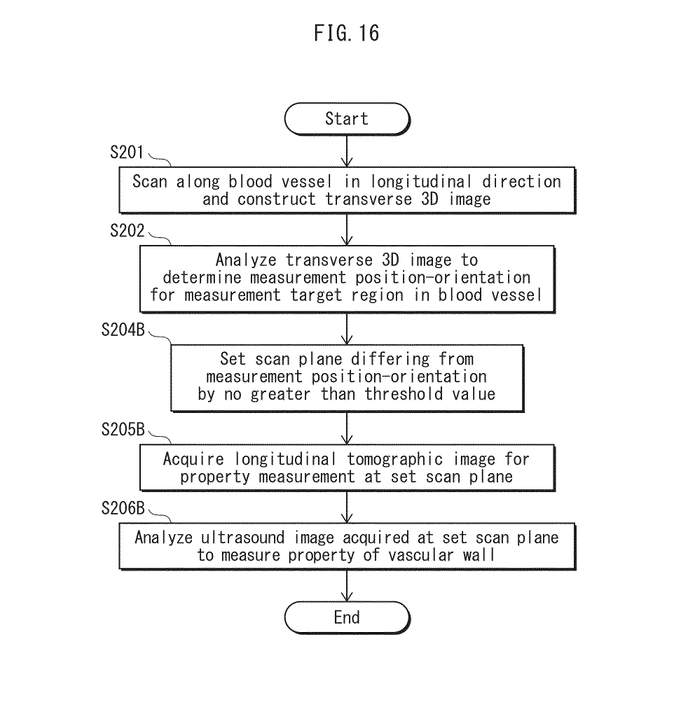

Operation of the ultrasound diagnostic apparatus 40, configured as described above, is explained below with reference to FIG. 16. FIG. 16 illustrates a flowchart for the ultrasound diagnostic apparatus 40 relating to the fourth embodiment.

(Step S201)

In Step S201, the transducer columns of the ultrasound probe 91 are positioned approximately perpendicular to the longitudinal direction of the carotid artery, and transducer columns which are driven are scanned along the carotid artery in the longitudinal direction to acquire a plurality of transverse tomographic images of the carotid artery. Subsequently, vascular contours are extracted from each of the transverse tomographic images. Based on positions and orientations of scan planes each corresponding to the one of the transverse tomographic images, the vascular contours are mapped in a 3D space in order to construct a 3D image of the carotid artery. During the above, a position and an orientation of each of the scan planes is calculated based on a column number, received from the ultrasound probe 91, for which a transverse tomographic image of the scan plane is acquired. Among the tunica intima, the tunica media and the tunica adventitia, when extracting vascular contours, contours are extracted for at least the tunica adventitia.

(Step S202)

Using the same method as in the first embodiment, in Step S202 shapes of contours of the tunica adventitia in the transverse 3D image are analyzed to determine a position and an orientation of a scan plane of the ultrasound probe 91 for acquiring a tomographic image including a measurement target region of the blood vessel. In order that IMT measurement is performed using a longitudinal tomographic image, a position and an orientation are acquired which are of a scan plane for acquiring a longitudinal tomographic image which includes the measurement target region.

(Step S204B)

In Step S204B a scan plane of the ultrasound probe 91 for acquiring the longitudinal tomographic image including the measurement target region for IMT is determined such as to differ from the measurement position-orientation information locRef by no greater than a threshold value. In order that the scan plane is formed parallel to the longitudinal direction, the scan plane is determined such that at least one of the transducer rows, which are parallel to the longitudinal direction, is driven.

(Step S205B)

In Step S205B the ultrasound probe 91 is driven to acquire the longitudinal tomographic image at the scan plane determined in Step S204B. In order to perform the above, the scan plane setting unit 408 instructs the transmission-reception processing unit 100 to perform transmission processing and reception processing to acquire an ultrasound image at the scan plane, which is a longitudinal tomographic image for property measurement.

(Step S206B)

In Step S206B, IMT measurement is performed in the range indicated by the measurement range information mesRan, based on the ultrasound image acquired in Step S205B, using the same method as in the first embodiment.

<Effects>

As explained above, the ultrasound diagnostic apparatus 40 can be used with the ultrasound probe 91 which has a configuration in which the plurality of transducer columns 91a, each having a plurality of ultrasound transducers arranged in the column direction X, are configured to scan in the row direction Y perpendicular to the column direction X. Thus, the ultrasound diagnostic apparatus 40 can easily set a scan plane 91y of the ultrasound probe 91 for acquiring a longitudinal tomographic image of a measurement target region for IMT in a blood vessel. Therefore, through the ultrasound diagnostic apparatus 40 the operator is always able to acquire the longitudinal tomographic image including the measurement target region, and thus can perform IMT measurement accurately.

Also, IMT measurement is performed based on the longitudinal tomographic image which is newly acquired at the scan plane 91y, at which the longitudinal tomographic image of the measurement target region for IMT can be acquired. Therefore, a problem does not occur of results of IMT measurement varying along the longitudinal direction in accordance with vascular pulsations, such as occurs when IMT measurement is performed based on a longitudinal cross-section generated from a transverse 3D image.

Furthermore, setting of the scan plane 91y of the ultrasound probe 91, at which the longitudinal tomographic image of the measurement target region for IMT in the blood vessel can be acquired, is performed automatically. Therefore, the operator is not required to move the ultrasound probe 91 and IMT measurement can be easily performed even if the operator does not possess a high degree of procedural skill.

### Modified Examples

The above is an explanation of the ultrasound diagnostic apparatus 40 relating to the present embodiment. The ultrasound diagnostic apparatus in the present disclosure is of course not limited to the ultrasound diagnostic apparatus explained in the present embodiment and various modified examples of the ultrasound diagnostic apparatus in the present embodiment are also possible, such as explained below.

(1) For the ultrasound diagnostic apparatus 40, the ultrasound probe 91 is explained using an example in which transducers 91c are arranged in a matrix as a 2D array consisting of a plurality of transducer columns 91a, each having a plurality of ultrasound transducers arranged in the column direction X, which are arranged in a row direction Y perpendicular to the column direction X. However, so long as the ultrasound probe is configured in a manner such that at least one transducer column thereof, having a plurality of ultrasound transducers arranged in a column direction, is configured to scan in a row direction perpendicular to the column direction, the ultrasound probe may alternatively be configured in a manner such that the transducer column is movable perpendicular to the column direction. For example, a 3D image may be acquired using a mechanical oscillating type ultrasound probe in which a 1D array of ultrasound transducers moves in an oscillating motion within the ultrasound probe.

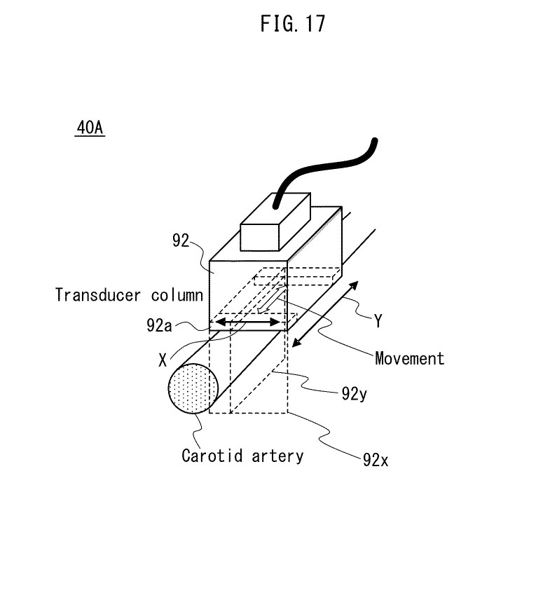

FIG. 17 is a schematic diagram illustrating an ultrasound probe 92 used by an ultrasound diagnostic apparatus 40A relating to a modified example of the ultrasound diagnostic apparatus 40 relating to the fourth embodiment. The ultrasound probe 92 is configured in a manner such that a transducer column 92a thereof, having a plurality of ultrasound transducers arranged in a column direction X, is moveable in a row direction Y perpendicular to the column direction X. The transducer column 92a moves in the row direction Y through a oscillating motion. A scan plane 92x can be formed parallel to the column direction X by driving the transducer column 92a. On the other hand, a scan plane 92y can be formed parallel to the row direction Y by driving at least one of the transducers in the transducer column 92a while moving the transducer column 92a in the row direction Y. A transverse 3D image is constructed from a plurality of transverse tomographic images acquired at scan planes 92x. A position and an orientation are determined at which a longitudinal tomographic image of a measurement target region for IMT can be acquired and, through the scan plane setting unit 408, a scan plane 92y can be set for acquiring the longitudinal tomographic image. Therefore, through the above configuration the operator can always acquire the longitudinal tomographic image including the measurement target region and thus can perform IMT measurement accurately.

(2) The present embodiment is explained using an example in which, in terms of a 3D probe for acquiring a 3D image, a matrix probe is used as an example of a 2D array probe having ultrasound transducers arranged in a 2D array on a probe surface thereof. However, the 2D array probe which is used is not limited to the type described above and alternatively a different type of probe may be selected such as a linear probe, convex probe or sector probe.

(3) In a situation in which an observation range of a 3D probe is narrow, scanning may be performed while moving the 3D probe and 3D images acquired at different positions of the 3D probe may be connected in order to acquire a 3D image over a wider range. In other words, when the carotid artery is greater in extent than a 3D region which can be acquired during a single scan using the 3D probe, by connecting 3D areas acquired through scanning a plurality of times, a 3D image can be acquired of the entire carotid artery. During the above, the 3D probe may be moved in a manner such that, among the 3D images acquired through the plurality of scans, neighboring 3D images overlap with one another. Furthermore, when connecting the 3D images, the 3D images may be connected so as to be continuous by matching positions of vascular contours in transverse cross-section images extracted from the 3D images or by matching central lines of the vascular contours.

### Fifth Embodiment

By recording on a recording medium such as a floppy disk, a program for implementing the control method for an ultrasound diagnostic apparatus described in each of the above embodiments, an independent computer system can easily execute processing described in each of the above embodiments.

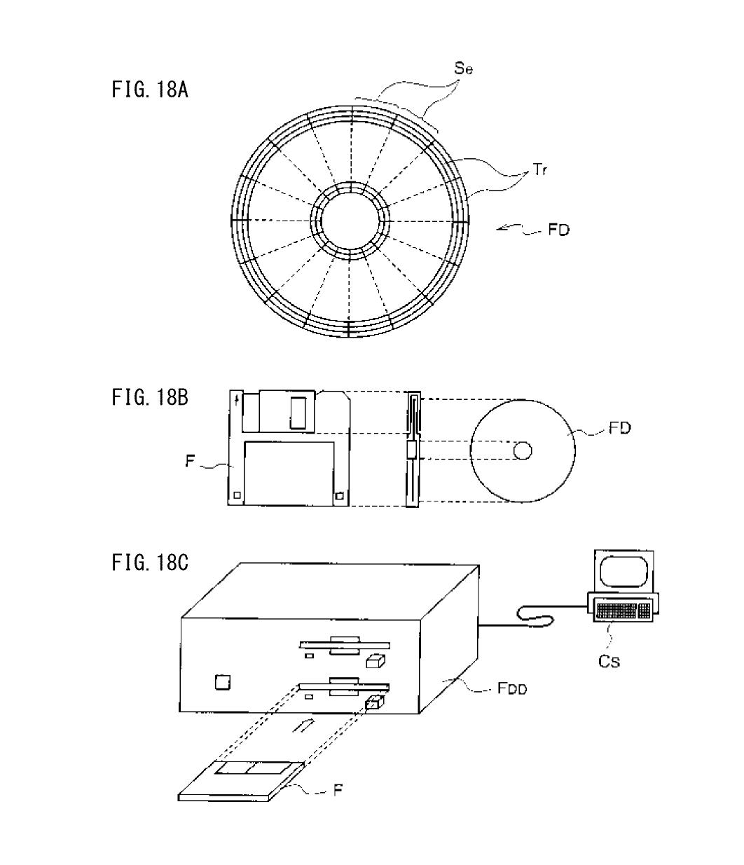

FIGS. 18A-18C are provided for explaining a configuration in which the control method for an ultrasound diagnostic apparatus described in each of the above embodiments is executed by a computer system using a program recorded on a recording medium such as a floppy disk.

FIG. 18A illustrates an example of physical format of a floppy disk FD, which is an example of a recording medium. FIG. 18B illustrates an external front view, a cross-section view and an internal view of the floppy disk FD. The floppy disk FD is housed in a case F. A plurality of tracks Tr are formed on a surface of the floppy disk FD in concentric circles from an outer circumference to an inner circumference of the floppy disk FD. Each track is divided into 16 sectors Se in terms of angle from a center of the floppy disk FD. Therefore, the above program can be stored on a floppy disk by recording the program in an allotted region on the floppy disk FD.

FIG. 18C illustrates a configuration for executing the program recorded on the floppy disk FD. When recording the program for implementing the control method of an ultrasound diagnostic apparatus on the floppy disk FD, a computer system Cs writes the program using a floppy disk drive. Furthermore, when constructing, in a computer system, the control method of an ultrasound diagnostic apparatus which is implemented by the program recorded on the floppy disk, the program is read from the floppy disk by the floppy disk drive and is transmitted to the computer system.

In the above explanation, a floppy disk is used as an example of a recording medium, however alternatively an optical disk may be used in the same way. The recording medium is not limited to being a floppy disk or an optical disk, and alternatively may be any medium on which a program can be recorded, such as an IC (Integrated Circuit) card or a ROM cassette.

Functional blocks in each of the ultrasound diagnostic apparatuses illustrated in FIGS. 1, 6, 11 and 14 may typically be implemented through large scale integration (LSI) as integrated circuits. Each of the functional blocks may be integrated into a chip or alternatively all or a portion of the functional blocks may be integrated into a single chip.

The above refers to LSI, but depending on the degree of integration the above may also be referred to as IC, system LSI, super LSI or ultra LSI.

Also, circuit integration is not limited to LSI and may alternatively be realized through a dedicated circuit or a general processor. For example, a dedicated circuit for graphics processing may be used such as a graphic processing unit (GPU). A field programmable gate array (FPGA), which is programmable after the LSI is manufactured, or a reconfigurable processor, which allows for reconfiguration of the connection and setting of circuit cells inside the LSI, may alternatively be used.

Furthermore, if technology for forming integrated circuits that replaces LSI were to emerge, owing to advances in semiconductor technology or to another derivative technology, the integration of functional blocks may naturally be accomplished using such technology. Application of biotechnology is also possible.

Furthermore, the configuration elements of each of the ultrasound diagnostic apparatuses illustrated in FIGS. 1, 6, 11 and 14 may be connected via a network such as the Internet or a local area network (LAN). For example, in an alternative configuration ultrasound images may be read from an accumulation device or a server in a network, which stores the ultrasound images therein. Additional functions of each of the configuration elements may also be performed through the network.

[Supplementary Explanation]

Each of the embodiments described above is a non-limiting and exemplary embodiment of the apparatus in the present disclosure. Numbers, shapes, materials, configuration elements, connection structure and position of the configuration elements, processes, orders of processes and the like described in the embodiments are merely non-limiting examples thereof. Configuration elements described in the embodiments but not recited in independent claims, which each indicate a general concept of the present disclosure, are explained as arbitrary configuration elements for purposes of configuring preferable embodiments.

Furthermore, in drawings referred to in the above embodiments, in order to facilitate understanding, configuration elements are not necessarily illustrated to scale. The present disclosure is not limited by the above embodiments, and appropriate modifications may be made so long as such modifications do not cause deviation from the general concept of the present disclosure.

In an ultrasound diagnostic apparatus, circuit components, leads and the like mounted on circuit boards are also present. Various different embodiments of electrical wiring and electrical circuits are possible based on common knowledge in the technical field of imaging diagnostic apparatuses, however as such embodiments are not directly relevant to explanation of the present disclosure, explanation thereof is omitted. The above drawings are schematic diagrams and do not necessarily provide entirely accurate illustrations.

### INDUSTRIAL APPLICABILITY

Through the ultrasound diagnostic apparatus and the control method thereof in the present disclosure, navigation information is provided to an operator in order that IMT of a blood vessel can be measured at a most suitable scan plane, and therefore IMT measurement can be easily performed with a high degree of reproducibility. Consequently, the ultrasound diagnostic apparatus and the control method thereof in the present disclosure improve diagnosis accuracy and reduce examination time, for example in screening for arteriosclerosis, and therefore are applicable for widespread use in the field of medical diagnostic apparatuses.

### REFERENCE SIGNS LIST

10, 20, 30, 40, 40A ultrasound diagnostic apparatus
80 display
90, 91, 92 ultrasound probe
100 transmission-reception processing unit
101 2D image generation unit
102 transverse 3D image construction unit
103 measurement position-orientation determination unit
104 probe position-orientation detection unit
104a image capture sub-unit
104b optical marker
105, 205 judgment unit
106, 206, 306 property measurement unit
107 display control unit
203a transverse information analysis sub-unit
203b longitudinal information analysis sub-unit
203c measurement position determination sub-unit
408 scan plane setting unit

1. An ultrasound diagnostic apparatus to which an ultrasound probe and a position-orientation detection unit, configured to detect a position and an orientation of the ultrasound probe, are connectable, the ultrasound diagnostic apparatus comprising:
a transmission-reception processing unit configured to transmit an ultrasound wave toward a blood vessel which is a measurement target via the ultrasound probe and to receive a reflected ultrasound wave from the blood vessel via the ultrasound probe;
a 2D image generation unit configured to generate a tomographic image of the blood vessel based on the reflected ultrasound wave;
a measurement position-orientation determination unit configured to determine a measurement target region of the blood vessel, based on a 3D image of the blood vessel generated from a plurality of transverse tomographic images acquired by scanning of the ultrasound probe along the blood vessel in a longitudinal direction, and to determine a measurement position and a measurement orientation of the ultrasound probe for acquiring a longitudinal tomographic image including the measurement target region;
a judgment unit configured to judge whether a current position and a current orientation of the ultrasound probe, detected by the position-orientation detection unit at a current time, differ from the measurement position and the measurement orientation respectively by no greater than a threshold value; and
a property measurement unit configured to calculate a property of a vascular wall of the blood vessel in the measurement target region, wherein
when the judgment unit judges that the current position and the current orientation differ from the measurement position and the measurement orientation respectively by no greater than the threshold value, the property measurement unit calculates the property of the vascular wall based on the longitudinal tomographic image of the blood vessel.

2. The ultrasound diagnostic apparatus of claim 1 to which a display is connectable, further comprising
a display control unit configured to control the display to display the 3D image of the blood vessel, the measurement position and the measurement orientation, and the current position and the current orientation of the ultrasound probe.

3. The ultrasound diagnostic apparatus of claim 1, further comprising
the position-orientation detection unit.

4. The ultrasound diagnostic apparatus of claim 1, further comprising
a transverse 3D image construction unit configured to construct the 3D image of the blood vessel based on the plurality of transverse tomographic images and based on position-orientation information indicating a position and an orientation of the ultrasound probe when each of the transverse tomographic images is acquired.

5. The ultrasound diagnostic apparatus of claim 1, wherein
the blood vessel is a carotid artery, and
the property of the vascular wall is an intima-media thickness.

6. The ultrasound diagnostic apparatus of claim 5, wherein
the measurement position-orientation determination unit determines the measurement target region for intima-media thickness based on a boundary position between a common carotid artery and a bulb of the common carotid artery, and determines the measurement position and the measurement orientation in a manner such that the measurement target region is included in a reception signal acquisition range of the ultrasound probe when the ultrasound probe is positioned at the measurement position and the measurement orientation.

7. The ultrasound diagnostic apparatus of claim 5, wherein
the measurement position-orientation determination unit detects a position of maximum hypertrophy, at which the intima-media thickness is a maximum value, in at least one part of the carotid artery, and determines the measurement position and the measurement orientation in a manner such that the position of maximum hypertrophy is included in a reception signal acquisition range of the ultrasound probe when the ultrasound probe is positioned at the measurement position and the measurement orientation, and
the part of the carotid artery is a common carotid artery, a bulb of the common carotid artery or an internal carotid artery.

8. The ultrasound diagnostic apparatus of claim 5, wherein
the measurement position-orientation determination unit detects a position of maximum hypertrophy, at which the intima-media thickness is a maximum value, in at least one part of the carotid artery,
the part of the carotid artery is a common carotid artery, a bulb of the common carotid artery or an internal carotid artery, and
the property measurement unit measures an intima-media volume in a region including the position of maximum hypertrophy, based on the 3D image of the blood vessel.

9. An ultrasound diagnostic apparatus to which an ultrasound probe and a position-orientation detection unit, configured to detect a position and an orientation of the ultrasound probe, are connectable, the ultrasound diagnostic apparatus comprising:
a transmission-reception processing unit configured to transmit an ultrasound wave toward a blood vessel which is a measurement target via the ultrasound probe and to receive a reflected ultrasound wave from the blood vessel via the ultrasound probe;
a 2D image generation unit configured to generate a tomographic image of the blood vessel based on the reflected ultrasound wave;
a measurement position-orientation determination unit configured to determine a measurement target region of the blood vessel, based on a plurality of transverse tomographic images acquired by scanning of the ultrasound probe along the blood vessel in a longitudinal direction and based on position-orientation information indicating a position and an orientation of the ultrasound probe when each of the transverse tomographic images is acquired, and to determine a measurement position and a measurement orientation of the ultrasound probe for acquiring a longitudinal tomographic image including the measurement target region;
a judgment unit configured to judge whether a current position and a current orientation of the ultrasound probe, detected by the position-orientation detection unit at a current time, differ from the measurement position and the measurement orientation respectively by no greater than a threshold value; and
a property measurement unit configured to calculate a property of a vascular wall of the blood vessel in the measurement target region, wherein
when the judgment unit judges that the current position and the current orientation differ from the measurement position and the measurement orientation respectively by no greater than the threshold value:
the transmission-reception unit transmits the ultrasound wave and receives the reflected ultrasound wave via the ultrasound probe which is positioned at the current position and the current orientation;
the 2D image generation unit generates the longitudinal tomographic image of the blood vessel based on the reflected ultrasound wave; and
the property measurement unit calculates the property of the vascular wall based on the longitudinal tomographic image of the blood vessel.

10. An ultrasound diagnostic apparatus to which an ultrasound probe and a position-orientation detection unit, configured to detect a position and an orientation of the ultrasound probe, are connectable, the ultrasound diagnostic apparatus comprising:
a transmission-reception processing unit configured to transmit an ultrasound wave toward a blood vessel which is a measurement target via the ultrasound probe and to receive a reflected ultrasound wave from the blood vessel via the ultrasound probe;
a 2D image generation unit configured to generate a tomographic image of the blood vessel based on the reflected ultrasound wave;
a transverse 3D image construction unit configured to construct a 3D image of the blood vessel based on a plurality of transverse tomographic images acquired by scanning of the ultrasound probe along the blood vessel in a longitudinal direction and based on position-orientation information indicating a position and an orientation of the ultrasound probe when each of the transverse tomographic images is acquired;
a transverse information analysis unit configured to determine, based on the 3D image of the blood vessel, a measurement position and a measurement orientation of the ultrasound probe for acquiring a longitudinal tomographic image for measuring a property of a vascular wall of the blood vessel;
a judgment unit configured to judge whether a current position and a current orientation of the ultrasound probe, detected by the position-orientation detection unit at a current time, differ from the measurement position and the measurement orientation respectively by no greater than a threshold value;
a longitudinal information analysis unit configured to determine an updated measurement position of the ultrasound probe for acquiring a longitudinal tomographic image including a measurement target region of the blood vessel;
a measurement position determination unit configured to determine the measurement target region of the blood vessel for measuring the property of the vascular wall, based on the updated measurement position; and
a property measurement unit configured to calculate the property of the vascular wall in the measurement target region based on the longitudinal tomographic image, wherein
when the judgment unit judges that the current position and the current orientation differ from the measurement position and the measurement orientation respectively by no greater than the threshold value:
the transmission-reception unit transmits the ultrasound wave and receives the reflected ultrasound wave via the ultrasound probe which is located at the current position and the current orientation;
the 2D image generation unit generates the longitudinal tomographic image of the blood vessel based on the reflected ultrasound wave;
the longitudinal information analysis unit determines the updated measurement position based on the longitudinal tomographic image;
the measurement position determination unit determines the measurement target region, for measuring the property of the vascular wall, based on the updated measurement position; and
the property measurement unit calculates the property of the vascular wall in the measurement target region based on the longitudinal tomographic image of the blood vessel.

11. An ultrasound diagnostic apparatus to which an ultrasound probe is connectable which is configured such that at least one transducer column thereof, having a plurality of ultrasound transducers arranged in a column, is configured to scan in a row direction perpendicular to the column, the ultrasound diagnostic apparatus comprising:
a transmission-reception processing unit configured to transmit an ultrasound wave toward a blood vessel which is a measurement target via the ultrasound probe and to receive a reflected ultrasound wave from the blood vessel via the ultrasound probe;
a 2D image generation unit configured to generate a tomographic image of the blood vessel based on the reflected ultrasound wave;
a measurement position-orientation determination unit configured to determine a measurement target region of the blood vessel for measuring a property of a vascular wall of the blood vessel based on a 3D image of vascular contours formed by arranging in a 3D space, vascular contours of the blood vessel extracted from a plurality of tomographic images generated by the 2D image generation unit, the tomographic images being acquired by scanning of the transducer column in the row direction along one direction of the blood vessel and the vascular contours being arranged based on each of the tomographic images and a row direction position of the transducer column when the tomographic image is acquired;
a scan plane setting unit configured to determine a column position of the ultrasound transducers for acquiring a tomographic image that is parallel to the row direction and that includes the measurement target region, and to instruct the transmission-reception processing unit to perform transmission processing and reception processing for acquiring a tomographic image for property measurement at the column position; and
a property measurement unit configured to measure the property of the vascular wall by analyzing the tomographic image for property measurement.

12. The ultrasound diagnostic apparatus of claim 11, wherein
the ultrasound probe is configured in a manner such that a plurality of transducer columns thereof, each having by a plurality of ultrasound transducers arranged in a column, are arranged in the row direction perpendicular to the column.

13. The ultrasound diagnostic apparatus of claim 11, wherein
the ultrasound probe is configured in a manner such that a transducer column thereof, having a plurality of ultrasound transducers arranged in a column, is configured to move in a direction perpendicular to the column.

14. A control method for an ultrasound diagnostic apparatus to which an ultrasound probe and a position-orientation detection unit, configured to detect a position and an orientation of the ultrasound probe, are connectable, the control method comprising:
a step of transmitting an ultrasound wave toward a blood vessel which is a measurement target via the ultrasound probe and receiving a reflected ultrasound wave from the blood vessel via the ultrasound probe;
a step of generating a tomographic image of the blood vessel based on the reflected ultrasound wave;
a step of determining a measurement target region of the blood vessel based on a 3D image of the blood vessel generated from a plurality of transverse tomographic images acquired by scanning of the ultrasound probe along the blood vessel in a longitudinal direction, and determining a measurement position and a measurement orientation of the ultrasound probe for acquiring a longitudinal tomographic image including the measurement target region;
a step of judging whether a current position and a current orientation of the ultrasound probe, detected by the position-orientation detection unit at a current time, differ from the measurement position and the measurement orientation respectively by no greater than a threshold value; and
a step of calculating a property of a vascular wall of the blood vessel in the measurement target region, based on the longitudinal tomographic image, when the current position and the current orientation of the ultrasound probe differ from the measurement position and the measurement orientation respectively by no greater than the threshold value.
